(19) United States
(12) Patent Application Publication
(10) Pub. No.: US 2025/0206747 A1
(43) Pub. Date: Jun. 26, 2025

(54) COELENTERAZINE ANALOGUES

(71) Applicant: Promega Corporation, Madison, WI (US)

(72) Inventors: Matthew A. Larsen, Madison, WI (US); Mark A. Klein, Madison, WI (US); Thomas Kirkland, Madison, WI (US)

(21) Appl. No.: 18/990,531

(22) Filed: Dec. 20, 2024

Related U.S. Application Data
(60) Provisional application No. 63/612,718, filed on Dec. 20, 2023.

(51) Int. Cl. C07D 487/04; C12Q 1/66

(52) U.S. Cl. CPC C07D 487/04; C12Q 1/66

(57) ABSTRACT

Described are coelenterazine analogue compounds, methods for making the compounds, kits comprising the compounds, and methods of using the compounds for the detection of luminescence in luciferase-based assays. For example, the compounds may be used for in vivo imaging.

### CROSS-REFERENCE TO RELATED APPLICATIONS

This application claims priority to and the benefit of U.S. Provisional Application No. 63/612,718, filed on Dec. 20, 2023, which is incorporated herein by reference in its entirety.

### TECHNICAL FIELD

Described are coelenterazine analogue compounds, methods for making the compounds, kits comprising the compounds, and methods of using the compounds for the detection of luminescence in luciferase-based assays. For example, the compounds may be used for in vivo imaging.

### BACKGROUND

Bioluminescent assays are used extensively in the investigation of cellular physiology, especially processes associated with gene expression. In particular, luciferase reporter enzymes are quite valuable tools in this field, and, to date, there has been intense protein engineering to obtain small and environmentally insensitive luciferases that may be useful in bioluminescent assays. There exist a number of efficient luciferase reporters enabling whole-cell biosensor measurements, drug discovery through high-throughput screening, and in vivo imaging, which also permits the study of protein-protein interactions in living cells, apoptosis, and cell viability. Luciferases that use coelenterazine and coelenterazine analogues as substrates are among the most widely used systems due to their brightness and acceptance in whole cell applications.

### SUMMARY

In one aspect, disclosed herein is a compound of formula (I):

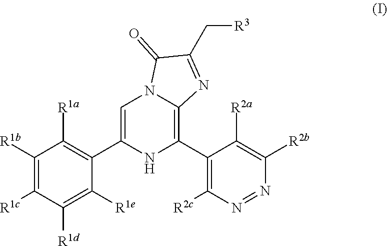

or a tautomer or a salt thereof, wherein:
R^{1a}, R^{1b}, R^{1c}, R^{1d}, R^{1e}, R^{2a}, R^{2b}, and R^{2c} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2} NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀ haloalkyl, hydroxy, cyano, nitro, amino, C₁₋₁₀alkoxy, aryl, heteroaryl, heterocycle, and C₃-6cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

In some embodiments, the compound is a compound of formula (I′):

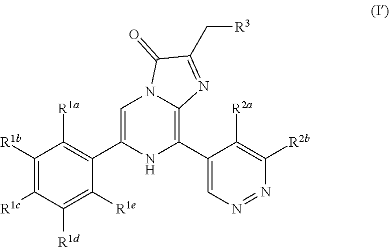

or a tautomer or a salt thereof, wherein:
R^{1a}, R^{1b}, R^{1c}, R^{1d}, R^{1e}, R^{2a}, and R^{2b} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2}, —NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀haloalkyl, hydroxy, cyano, nitro, amino, C₁₋₁₀alkoxy, aryl, heteroaryl, heterocycle, and C₃₋₆cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

In some embodiments, R^{1a}, R^{1b}, R^{1c}, R^{1d}, R^{1e}, R^{2a}, R^{2b}, and R^{2c} are each independently selected from hydrogen, halogen, —CN, C₁₋₄alkyl, C₁₋₄haloalkyl, —OR^{x1}, and —NR^{x1}R^{x2}, wherein R^{x1} and R^{x2} are independently selected from hydrogen and C₁₋₄alkyl. In some embodiments, wherein R^{1a}, R^{1b}, R^{1c}, R^{1d}, R^{1e}, R^{2a}, and R^{2b} are each independently selected from hydrogen, halogen, —CN, C₁₋₄alkyl, C₁₋₄haloalkyl, —OR^{x1}, and —NR^{x1}R^{x2}, wherein R^{x1} and R^{x2} are independently selected from hydrogen and C₁₋₄alkyl.

In some embodiments, R^{1a} is hydrogen or halogen. In some embodiments, R^{1a} is hydrogen. In some embodiments, R^{1a} is halogen. In some embodiments, R^{1a} is fluoro.

In some embodiments, R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl. In some embodiments, R^{1b} is hydrogen, methyl, fluoro, difluoromethyl, methoxy, —OH, or —NH₂.

In some embodiments, R^{1b} is hydrogen, —OR^{x1}, or —NR^{x1}R^{x2}, wherein R^{x1} and R^{x2} are independently selected from hydrogen and methyl. In some embodiments, R^{1b} is hydrogen, —OH, or —NH₂. In some embodiments, R^{1b} is halo. In some embodiments, R^{1b} is fluoro In some embodiments, R^{1b} is hydrogen. In some embodiments, R^{1b} is —OH. In some embodiments, R^{1b} is —NH₂. In some embodiments, R^{1b} is C₁₋₄alkyl. In some embodiments, R^{1b} is methyl. In some embodiments, R^{1b} is C₁₋₄haloalkyl. In some embodiments, R^{1b} is difluoromethyl.

In some embodiments, R^{2b} is hydrogen, halogen, —CN, or C₁₋₁₀alkyl. In some embodiments, R^{2b} is halogen, —CN, or methyl. In some embodiments, R^{2b} is halogen. In some embodiments, R^{2b} is chloro.

In some embodiments, R^{1c}, R^{1d}, R^{1e}, R^{2a}, and R^{2c} are each hydrogen. In some embodiments, R^{1c}, R^{1d}, R^{1e}, and R^{2a} are each hydrogen.

In some embodiments, R³ is phenyl or a monocyclic 5- or 6-membered heteroaryl having 1 or 2 heteroatoms independently selected from O, S, and N. In some embodiments, R³ is phenyl, a monocyclic 5-membered heteroaryl having 1 heteroatom selected from O, S, and N, or a monocyclic 6-membered heteroaryl having 1 or 2 N atoms. In some embodiments, R³ is phenyl, furanyl, or pyridinyl. In some embodiments, R³ is phenyl or a monocyclic 5-membered heteroaryl having 1 heteroatom selected from O, S, and N. In some embodiments, R³ is furan-2-yl. In some embodiments, R³ is unsubstituted or substituted with 1 or 2 substituents selected from C₁₋₄alkyl, C₁₋₄haloalkyl, C₁₋₄alkoxy, halo, and hydroxy. In some embodiments, R³ is unsubstituted or substituted with 1 substituent selected from fluoro and methoxy. In some embodiments, R³ is unsubstituted or substituted with 1 substituent selected from C₁₋₄alkyl C₁₋₄haloalkyl, and hydroxy. In some embodiments, R³ is unsubstituted or substituted with 1 substituent selected from methyl, and trifluoromethyl. In some embodiments, R³ is unsubstituted.

In some embodiments, the compound is a compound of formula (Ia):

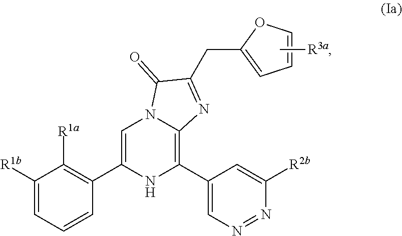

or a tautomer or a salt thereof, wherein:
R^{1a}, R^{1b}, and R^{2b} are each independently selected from hydrogen, halogen, —OR^{x1}, and —NR^{x1}R^{x2}, wherein R^{x1} and R^{x2} are each independently selected from hydrogen and C₁₋₄alkyl; and
R^{3a} is hydrogen, halogen, C₁₋₄alkyl, or C₁₋₄haloalkyl.

In some embodiments, R^{1a} is hydrogen or halo.

In some embodiments, R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl. In some embodiments, R^{1b} is hydrogen, methyl, fluoro, difluoromethyl, methoxy, —OH, or —NH₂.

In some embodiments, R^{2b} is halogen. In some embodiments, R^{2b} is chloro.

In some embodiments, R^{3a} is hydrogen.

In some embodiments, the compound is a compound of formula (Ib):

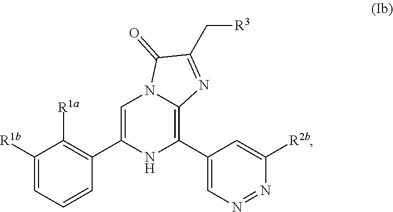

or a tautomer or a salt thereof, wherein:
R^{1a}, R^{1b}, and R^{2b} are each independently selected from hydrogen, C₁₋₄alkyl, C₁₋₄haloalkyl, halogen, —OR^{x1}, and —NR^{x1}R^{x2}, wherein R^{x1} and R^{x2} are each independently selected from hydrogen and C₁₋₄alkyl; and
R³ is phenyl or a monocyclic 5- or 6-membered heteroaryl having 1 or 2 heteroatoms independently selected from O, S, and N, wherein R³ is unsubstituted or substituted with 1 substituent selected from halo and C₁₋₄alkoxy.

In some embodiments, R^{1a} is hydrogen or halo.

In some embodiments, R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl. In some embodiments, R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl. In some embodiments, R^{1b} is hydrogen, methyl, fluoro, difluoromethyl, methoxy, —OH, or —NH₂.

In some embodiments, R^{2b} is halogen. In some embodiments, R^{2b} is chloro.

In some embodiments, R³ is phenyl, a monocyclic 5-membered heteroaryl having 1 heteroatom selected from O, S, and N, or a monocyclic 6-membered heteroaryl having 1 or 2 N atoms. In some embodiments, R³ is phenyl, furanyl, or pyridinyl. In some embodiments, R³ is unsubstituted or substituted with 1 substituent selected from halo and C₁₋₄alkoxy. In some embodiments, R³ is unsubstituted or substituted with 1 substituent selected from fluoro and methoxy. In some embodiments, R³ is unsubstituted.

In some embodiments, the compound of formula (I) is selected from the group consisting of:

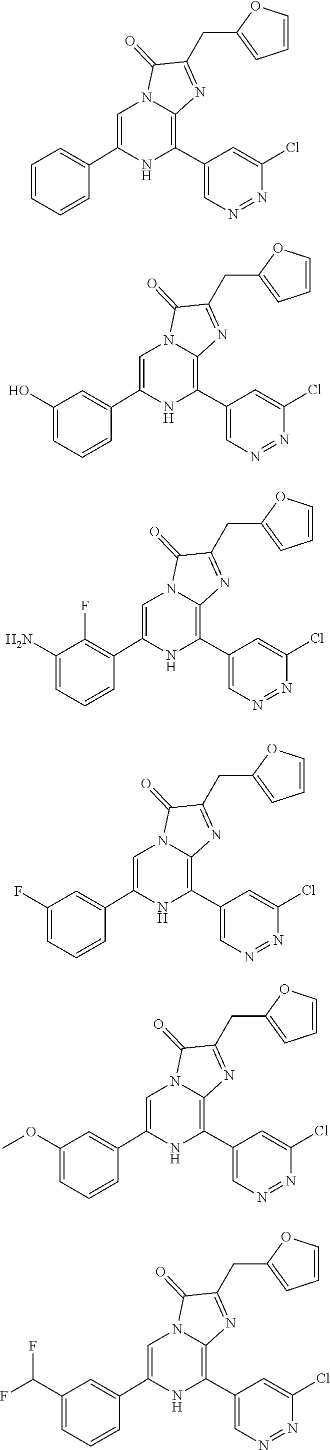

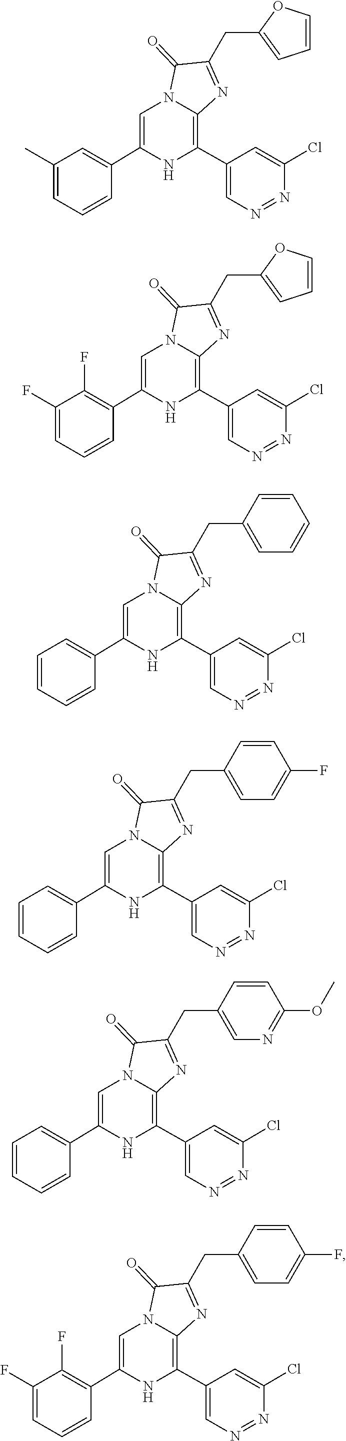

and tautomers and salts thereof.

In another aspect, disclosed herein is a kit comprising a compound of formula (I), or a tautomer or a salt thereof. In some embodiments, the kit further comprises a luciferase. In some embodiments, the kit further comprises a buffer reagent. In some embodiments, the kit further comprises instructions for performing a luminescence assay.

In another aspect, disclosed herein is a method for detecting luminescence in a sample, the method comprising: contacting a sample with a compound of formula (I), or a tautomer or a salt thereof; contacting the sample with a coelenterazine-utilizing luciferase, if it is not present in the sample; and detecting luminescence. In some embodiments, the sample contains live cells. In some embodiments, the sample contains a coelenterazine-utilizing luciferase.

In another aspect, disclosed herein is a method for detecting luminescence in a transgenic animal comprising: administering the compound of formula (I), or a tautomer or a salt thereof, to a transgenic animal; and detecting luminescence; wherein the transgenic animal expresses a coelenterazine-utilizing luciferase.

### BRIEF DESCRIPTION OF THE DRAWINGS

FIGS. 1A-1B show kinetic and spectral properties of coelenterazine analogues. (FIG. 1A) Michaelis-Menten analysis determining the relative luminescence produced by 0.01 nM NANOLUC® luciferase at various concentrations of coelenterazine analogues. These data were used to determine the Vmax values depicted in Scheme 1. (FIG. 1B) Luminescent spectra of 0.5 nM NANOLUC® luciferase in the presence of 20 μM coelenterazine analogue. These data were used to determine the peak emission (λem) depicted in Scheme 1.

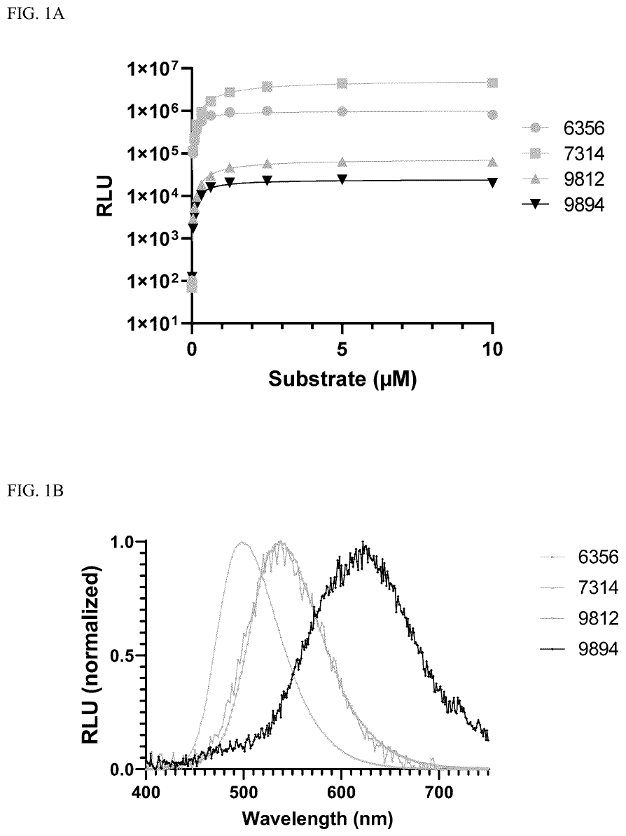

FIG. 2 shows a Michaelis-Menten analysis determining the relative luminescence produced by 0.01 nM NANOLUC® luciferase at various concentrations of coelenterazine analogues. These data were used to determine the Vmax values depicted in Table 1.

FIG. 3 shows a Michaelis-Menten analysis determining the relative luminescence produced by 0.01 nM NANOLUC® luciferase at various concentrations of coelenterazine analogues. These data were used to determine the Vmax values depicted in Table 2.

### DETAILED DESCRIPTION

Disclosed herein are coelenterazine analogues, which are useful substrates for proteins that utilize coelenterazine (“coelenterazine-utilizing enzymes”) to produce luminescence, including, but not limited to, luciferases and photoproteins found in various marine organisms such as cnidarians (e.g., Renilla luciferase), jellyfish (e.g., aequorin from the Aequorea jellyfish) and decapods luciferases (e.g., luciferase complex of Oplophorus gracilirostris).

In some embodiments, upon reaction of the disclosed compounds with an Oplophorus or Oplophorus-derived luciferase, compounds disclosed herein display significantly red-shifted emission compared to furimazine. Shifting the emission wavelength of bioluminescence has many advantages. For example, multiplexing of bioluminescence is useful for biochemical assays, antibody-based assays, cell-based assays, and in vivo imaging. Using red-shifted substrates, such as those disclosed herein, allows multiple colors to be obtained from the same enzyme either by switching substrates or by adding a mixture of substrates. This allows measurement of multiple parameters simultaneously, either at a defined point in time (e.g., an end point assay) or over time with multiple measurements (e.g., a live cell assay). Such multiplexing can be in the form of a plate based assay, an imaging assay, a rapid test (such as a lateral flow strip), and many other types of assays.

Red-shifted substrates also possess advantages as bioluminescent resonance energy transfer (BRET) donors. Since the magnitude of energy transfer is proportional to the overlap of the emission spectrum of the donor and the excitation spectrum of the acceptor according to the Forster equation, red-shifting the emission through use of red-shifted substrates allows better energy transfer to red, far-red, and infrared acceptors. This is advantageous for further multiplexing as well as tissue penetration of the emitted light.

In addition to these advantages, the structural modifications in red-shifted substrates described herein may possess enhanced stability, solubility, and biodistribution, as well as reduced toxicity or autoluminescence, under relevant assay or imaging conditions. These advantages are quite important for nearly all types of assay.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkylene” refers to a divalent group derived from a straight or branched saturated chain hydrocarbon, for example, of 1 to 6 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” refers to a group —NRₓR_{y}, wherein Rₓ and R_{y} are selected from hydrogen and alkyl (e.g., C₁-C₄ alkyl). A group —NH(alkyl) may be referred to herein as “alkylamino” and a group —N(alkyl)₂ may be referred to herein as “dialkylamino.”

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl). In some embodiments, an aryl group has fourteen ring carbon atoms (“C₁₄ aryl,” e.g., anthracenyl and phenanthrenyl).

As used herein, the term “cyano” refers to a group —CN.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen (“perhaloalkyl”). Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “haloalkylene” refers to an alkylene group, as defined herein, in which at least one hydrogen atom is replaced with a halogen.

As used herein, the term “haloalkoxy” refers to a haloalkyl group, as defined herein, appended to the parent molecular moiety through an oxygen atom. Representative examples of haloalkoxy include, but are not limited to, difluoromethoxy, trifluoromethoxy, and 2,2,2-trifluoroethoxy.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “hydroxy” or “hydroxyl” refers to an —OH group.

As used herein, the term “nitro” refers to an —NO₂ group.

As used herein, the term “oxo” refers to a group ═O.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

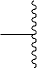

represents a point of attachment of one moiety to another moiety (e.g., a substituent group to the rest of the compound).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

When substituent groups are specified by their conventional chemical formulae, written from left to right, such indication also encompasses substituent groups resulting from writing the structure from right to left. For example, if a bivalent group is shown as —CH₂O—, such indication also encompasses —OCH₂—; similarly, —OC(O)NH— also encompasses —NHC(O)O—.

An “animal” as used herein includes, but is not limited to, mammals, amphibians, birds, fish, insects, reptiles, etc. Mammals can include, but are not limited, to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like. In some embodiments, the animal can be a human or a non-human. Suitable animals include both males and females and animals of any age, including embryonic (e.g., in utero or in ovo), infant, juvenile, adolescent, adult and geriatric animals.

“Fusion protein” and “fusion polypeptide” as used herein refers to a fusion comprising at least one bioluminescent protein in combination with a heterologous protein of interest, such as a fluorescent protein, as part of a single continuous chain of amino acids, which chain does not occur in nature.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Transgene” as used herein refers to a gene or genetic material containing a gene sequence that has been isolated and/or manipulated from one organism and is introduced into a different organism. The transgene may contain a transgenic sequence or a native or wild-type DNA sequence. This non-native segment of DNA can retain the ability to produce RNA or protein in the transgenic organism. For example, the transgene can encode a fusion protein, such as a fusion protein comprising a luciferase. A transgenic sequence can be partly or entirely species-heterologous, i.e., the transgenic sequence, or a portion thereof, can be from a species which is different from the cell into which it is introduced.

A “transgenic animal” refers to a genetically engineered animal or offspring of genetically engineered animals. A transgenic animal usually contains genetic material from at least one unrelated organism, such as from a virus, plant, or other animal.

The terms “transformation,” “transfection,” and “transduction” as used interchangeably herein refer to the introduction of a heterologous nucleic acid molecule, such as genetic material, into a cell. Such introduction into a cell can be stable or transient. Thus, in some embodiments, a host cell or host organism is stably transformed with a heterologous nucleic acid molecule, such as genetic material. In other embodiments, a host cell or host organism is transiently transformed with a heterologous nucleic acid molecule, such as genetic material. “Transient transformation” in the context of a polynucleotide means that a polynucleotide is introduced into the cell and does not integrate into the genome of the cell. By “stably introducing” or “stably introduced” in the context of a polynucleotide introduced into a cell is intended that the introduced polynucleotide is stably incorporated into the genome of the cell, and thus the cell is stably transformed with the polynucleotide. “Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear, the plasmid and the plastid genome, and therefore includes integration of the nucleic acid construct into, for example, the chloroplast or mitochondrial genome. Stable transformation as used herein can also refer to a transgene that is maintained extrachromasomally, for example, as a minichromosome or a plasmid. In some embodiments, the nucleotide sequences, constructs, expression cassettes can be expressed transiently and/or they can be stably incorporated into the genome of the host organism.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds

Disclosed herein is a compound of formula (I):

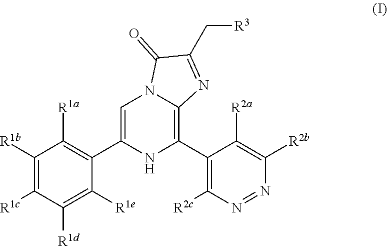

or a tautomer or a salt thereof, wherein:
R^{1a}, R^{1b}, R^{1c}, R^{1d}, R^{1e}, R^{2a}, R^{2b}, and R^{2c} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2}, —NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀haloalkyl, hydroxy, cyano, nitro, amino, C₁₋₁₀alkoxy, aryl, heteroaryl, heterocycle, and C₃₋₆cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

Also disclosed herein is a compound of formula (I′):

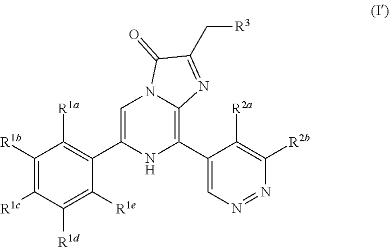

or a tautomer or a salt thereof, wherein:
R^{1a}, R^{1b}, R^{1c}, R^{1d}, R^{1e}, R^{2a}, and R^{2b} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2}, NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀haloalkyl, hydroxy, cyano, nitro, amino, C₁₋₁₀alkoxy, aryl, heteroaryl, heterocycle, and C₃₋₆cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

In some embodiments, R^{1a}, R^{1b}, R^{1c}, R^{1d}, R^{1e}, R^{2a}, R^{2b}, and R^{2c} are each independently selected from hydrogen, halogen, —CN, C₁₋₄alkyl, C₁₋₄haloalkyl, —OR^{x1}, and —NR^{x1}R^{x2}, wherein R^{x1} and R^{x2} are independently selected from hydrogen and C₁₋₄alkyl. In some embodiments, R^{1a}, R^{1b}, R^{1c}, R^{1d}, R^{1e}, R^{2a}, and R^{2b} are each independently selected from hydrogen, halogen, —CN, C₁₋₄alkyl, C₁₋₄haloalkyl, —OR^{x1}, and —NR^{x1}R^{x2}, wherein R^{x1} and R^{x2} are independently selected from hydrogen and C₁₋₄alkyl.

In some embodiments, R^{1a} is hydrogen or halogen. In some embodiments, R^{1a} is hydrogen. In some embodiments, R^{1a} is halogen. In some embodiments, R^{1a} is fluoro.

In some embodiments, R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl. In some embodiments, R^{1b} is hydrogen, —OR^{x1}, or —NR^{x1}R^{x2}, wherein R^{x1} and R^{x2} are independently selected from hydrogen and methyl. In some embodiments, R^{1b} is hydrogen, methyl, fluoro, difluoromethyl, methoxy, —OH, or —NH₂. In some embodiments, R^{1b} is hydrogen, —OH, or —NH₂. In some embodiments, R^{1b} is hydrogen. In some embodiments, R^{1b} is-OR^{x1}, wherein R^{x1} and R^{x2} are independently selected from hydrogen and methyl. In some embodiments, R^{1b} is —OH. In some embodiments, R^{1b} is methoxy. In some embodiments, R^{1b} is —NH₂. In some embodiments, R^{1b} is C₁₋₄alkyl. In some embodiments, R^{1b} is methyl. In some embodiments, R^{1b} is halo. In some embodiments, R^{1b} is fluoro. In some embodiments, R^{1b} is C₁₋₄haloalkyl. In some embodiments, R^{1b} is difluoromethyl.

In some embodiments, R^{1c} is hydrogen. In some embodiments, R^{1d} is hydrogen. In some embodiments, R^{1e} is hydrogen.

In some embodiments, R^{2a} is hydrogen.

In some embodiments, R^{2c} is hydrogen.

In some embodiments, R^{1c}, R^{1d}, R^{1e}, R^{2a}, and R^{2c} are each hydrogen. In some embodiments, R^{1c}, R^{1d}, R^{1e}, and R^{2a} are each hydrogen.

In some embodiments, R^{2b} is hydrogen, halogen, —CN, or C₁₋₁₀alkyl. In some embodiments, R^{2b} is halogen, —CN, or methyl. In some embodiments, R^{2b} is halogen. In some embodiments, R^{2b} is chloro.

In some embodiments, R³ is phenyl or a monocyclic 5- or 6-membered heteroaryl having 1 or 2 heteroatoms independently selected from O, S, and N. In some embodiments, R³ is phenyl, a monocyclic 5-membered heteroaryl having 1 heteroatom selected from O, S, and N, or a monocyclic 6-membered heteroaryl having 1 or 2 N atoms. In some embodiments, R³ is phenyl, furanyl, or pyridinyl. In some embodiments, R³ is phenyl or a monocyclic 5-membered heteroaryl having 1 heteroatom selected from O, S, and N. In some embodiments, R³ is phenyl. In some embodiments, R³ is furanyl. In some embodiments, R³ is furan-2-yl. In some embodiments, R³ is pyridinyl. In some embodiments, R³ is pyridin-3-yl.

In some embodiments, R³ is unsubstituted or substituted with 1 or 2 substituents selected from C₁₋₄alkyl, C₁₋₄haloalkyl, C₁₋₄alkoxy, halo, and hydroxy. In some embodiments, R³ is unsubstituted or substituted with 1 or 2 substituents selected from C₁₋₄alkyl, C₁₋₄haloalkyl, and hydroxy. In some embodiments, R³ is unsubstituted or substituted with 1 substituent selected from C₁₋₄alkyl, C₁₋₄haloalkyl, C₁₋₄alkoxy, halo, and hydroxy. In some embodiments, R³ is unsubstituted or substituted with 1 substituent selected from C₁₋₄alkyl, C₁₋₄haloalkyl, and hydroxy. In some embodiments, R³ is unsubstituted or substituted with 1 substituent selected from methyl, trifluoromethyl, methoxy, fluoro, and hydroxy. In some embodiments, R³ is unsubstituted or substituted with 1 substituent selected from methyl and trifluoromethyl. In some embodiments, R³ is unsubstituted or substituted with 1 substituent selected from halo and C₁₋₄alkoxy. In some embodiments, R³ is unsubstituted or substituted with 1 substituent selected from fluoro and methoxy. In some embodiments, R³ is unsubstituted.

In some embodiments, the compound of formula (I) is a compound of formula (Ia):

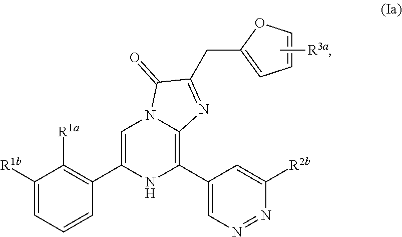

or a tautomer or a salt thereof, wherein:
R^{1a}, R^{1b}, and R^{2b} are each independently selected from hydrogen, halogen, —OR^{x1}, and —NR^{x1}R^{x2}, wherein R^{x1} and R^{x2} are each independently selected from hydrogen and C₁₋₄alkyl; and
R^{3a} is hydrogen, halogen, C₁₋₄alkyl, or C₁₋₄haloalkyl.

In some embodiments, the compound of formula (I) is a compound of formula (Ib):

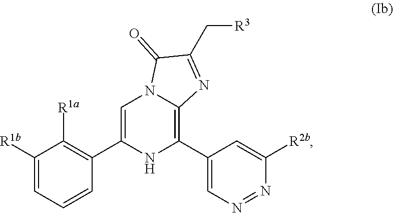

or a tautomer or a salt thereof, wherein:
R^{1a}, R^{1b}, and R^{2b} are each independently selected from hydrogen, C₁₋₄alkyl, C₁₋₄haloalkyl, halogen, —OR^{x1}, and —NR^{x1}R^{x2}, wherein R^{x1} and R^{x2} are each independently selected from hydrogen and C₁₋₄alkyl; and
R³ is phenyl or a monocyclic 5- or 6-membered heteroaryl having 1 or 2 heteroatoms independently selected from O, S, and N, wherein R³ is unsubstituted or substituted with 1 substituent selected from halo and C₁₋₄alkoxy.

In some embodiments, R^{1a} is selected from hydrogen and halo. In some embodiments, R^{1a} is selected from hydrogen and fluoro. In some embodiments, R^{1b} is hydrogen, C₁₋₄alkyl, C₁₋₄ haloalkyl, halogen, —OR^{x1}, or —NR^{x1}R^{x2}, wherein R^{x1} and R^{x2} are independently selected from hydrogen and methyl. In some embodiments, R^{1b} is hydrogen, methyl, fluoro, difluoromethyl, methoxy, —OH, or —NH₂.

In some embodiments, R^{2b} is halogen. In some embodiments, R^{2b} is chloro.

R³ is phenyl, a monocyclic 5-membered heteroaryl having 1 heteroatom selected from O, S, and N, or a monocyclic 6-membered heteroaryl having 1 or 2 N atoms. In some embodiments, R³ is phenyl, furanyl, or pyridinyl. In some embodiments, R³ is phenyl, furan-2-yl, or pyridine-3-yl. In some embodiments, R³ is unsubstituted or substituted with 1 substituent selected from halo and C₁₋₄alkoxy. In some embodiments, R³ is unsubstituted or substituted with 1 substituent selected from fluoro and methoxy. In some embodiments, R³ is unsubstituted.

In some embodiments, the compound is selected from the group consisting of:

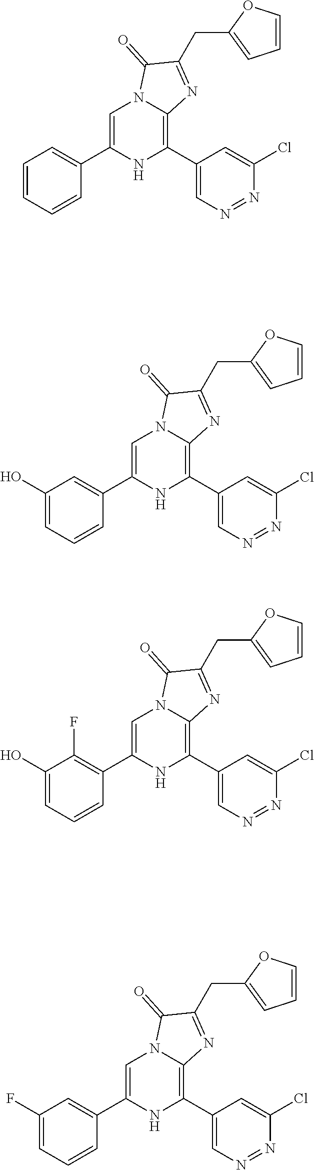 [SMILES: O=c1c(Cc2ccco2)nc2c(-c3cnnc(Cl)c3)[nH]c(-c3ccccc3)cn1-2 ; O=c1c(Cc2ccco2)nc2c(-c3cnnc(Cl)c3)[nH]c(-c3cccc(O)c3)cn1-2 ; O=c1c(Cc2ccco2)nc2c(-c3cnnc(Cl)c3)[nH]c(-c3cccc(O)c3F)cn1-2 ; O=c1c(Cc2ccco2)nc2c(-c3cnnc(Cl)c3)[nH]c(-c3cccc(F)c3)cn1-2]

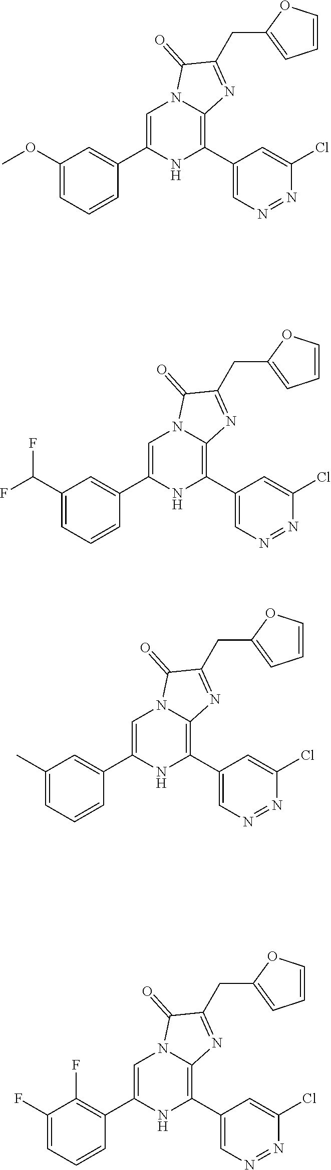 [SMILES: COc1cccc(-c2cn3c(=O)c(Cc4ccco4)nc-3c(-c3cnnc(Cl)c3)[nH]2)c1 ; O=c1c(Cc2ccco2)nc2c(-c3cnnc(Cl)c3)[nH]c(-c3cccc(C(F)F)c3)cn1-2 ; Cc1cccc(-c2cn3c(=O)c(Cc4ccco4)nc-3c(-c3cnnc(Cl)c3)[nH]2)c1 ; O=c1c(Cc2ccco2)nc2c(-c3cnnc(Cl)c3)[nH]c(-c3cccc(F)c3F)cn1-2]

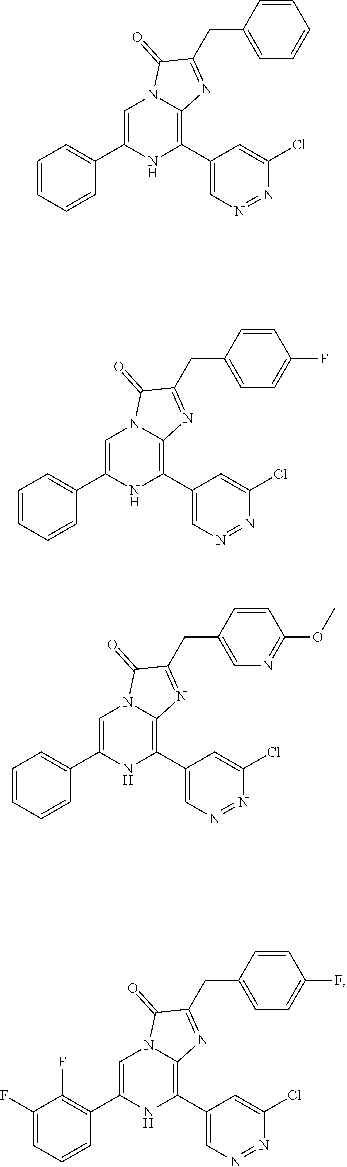 [SMILES: O=c1c(Cc2ccccc2)nc2c(-c3cnnc(Cl)c3)[nH]c(-c3ccccc3)cn1-2 ; O=c1c(Cc2ccc(F)cc2)nc2c(-c3cnnc(Cl)c3)[nH]c(-c3ccccc3)cn1-2 ; COc1ccc(Cc2nc3c(-c4cnnc(Cl)c4)[nH]c(-c4ccccc4)cn-3c2=O)cn1 ; O=c1c(Cc2ccc(F)cc2)nc2c(-c3cnnc(Cl)c3)[nH]c(-c3cccc(F)c3F)cn1-2]

and tautomers and salts thereof.

In some embodiments, the compounds may exist as stereoisomers wherein asymmetric or chiral centers are present. The stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof, and these are specifically included within the scope of this invention. Stereoisomers include enantiomers and diastereomers and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogel's Textbook of Practical Organic Chemistry”, 5^{th} edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds may possess tautomeric forms as well as geometric isomers, and that these also constitute an aspect of the invention. A compound of the invention or a tautomer or a salt thereof includes: the compound, salts of the compound, tautomers of the compound, and tautomers of the salts of the compound.

The present disclosure also includes isotopically-labeled compounds, which are identical to those recited in formula (I) or the specific compounds illustrated herein, but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain advantages resulting from greater metabolic stability, for example increased in vivo half-life or reduced dosage requirements, and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in the compounds are ¹¹C, ¹³N, ¹⁵O, and ¹⁸F. Isotopically-labeled compounds can generally be prepared by conventional techniques known to those skilled in the art or by processes analogous to those described in the accompanying Examples using appropriate isotopically-labeled reagent in place of non-isotopically-labeled reagent.

A compound described herein can be in the form of a salt. The selection of salts suitable for use in contact with the tissues of humans and lower animals without undue toxicity, irritation, allergic response, and the like, and are commensurate with a reasonable benefit/risk ratio is within the scope of sound medical judgement. Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Acceptable salts of the compounds of this invention include those derived from suitable inorganic and organic acids and bases. Examples of acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### A. Properties of the Compounds

The disclosed compounds may be substrates of luciferases to produce luminescence. The compounds may have improved water solubility, improved stability, improved cell permeability, increased biocompatibility with cells, reduced autoluminescence, and/or reduced toxicity.

“Luminescence” refers to the light output of a luciferase under appropriate conditions, e.g., in the presence of a suitable substrate such as a coelenterazine analogue. The light output may be measured as an instantaneous or near-instantaneous measure of light output (which is sometimes referred to as “T=0” luminescence or “flash”) at the start of the luminescence reaction, which may be initiated upon addition of the coelenterazine substrate. The luminescence reaction in various embodiments is carried out in a solution. In other embodiments, the luminescence reaction is carried out on a solid support. The solution may contain a lysate, for example from the cells in a prokaryotic or eukaryotic expression system. In other embodiments, expression occurs in a cell-free system, or the luciferase protein is secreted into an extracellular medium, such that, in the latter case, it is not necessary to produce a lysate. In some embodiments, the reaction is started by injecting appropriate materials, e.g., coelenterazine analogue, buffer, etc., into a reaction chamber (e.g., a well of a multiwell plate such as a 96-well plate) containing the luminescent protein. In still other embodiments, the luciferase and/or coelenterazine analogues (e.g., compounds disclosed herein) are introduced into a host, and measurements of luminescence are made on the host or a portion thereof, which can include a whole organism or cells, tissues, explants, or extracts thereof. The reaction chamber may be situated in a reading device which can measure the light output, e.g., using a luminometer or photomultiplier. The light output or luminescence may also be measured over time, for example in the same reaction chamber for a period of seconds, minutes, hours, etc. The light output or luminescence may be reported as the average over time, the half-life of decay of signal, the sum of the signal over a period of time, or the peak output. Luminescence may be measured in Relative Light Units (RLUs).

In some embodiments, disclosed compounds can have an RLU of greater than or equal to 1, greater than or equal to 2, greater than or equal to 3, greater than or equal to 4, greater than or equal to 5, greater than or equal to 10, greater than or equal to 20, greater than or equal to 30, greater than or equal to 40, greater than or equal to 50, or greater than or equal to 100, relative to coelenterazine or a known coelenterazine analogue such as furimazine.

In some embodiments, disclosed compounds can have a λmax of 450-750 nanometers, 450-700 nanometers, 460-650 nanometers, 470-650 nanometers, 480-650 nanometers, 490-650 nanometers, 500-650 nanometers, 510-650 nanometers, 520-650 nanometers, 530-650 nanometers, 540-650 nanometers, 550-650 nanometers, 560-650 nanometers, 570-650 nanometers, 580-650 nanometers, or 590-650 nanometers. Compounds disclosed herein can have a λmax greater than or equal to 450 nanometers, greater than or equal to 460 nanometers, greater than or equal to 470 nanometers, greater than or equal to 480 nanometers, greater than or equal to 490 nanometers, greater than or equal to 500 nanometers, greater than or equal to 510 nanometers, greater than or equal to 520 nanometers, greater than or equal to 530 nanometers, greater than or equal to 540 nanometers, greater than or equal to 550 nanometers, greater than or equal to 560 nanometers, greater than or equal to 570 nanometers, greater than or equal to 580 nanometers, greater than or equal to 590 nanometers, greater than or equal to 600 nanometers, greater than or equal to 610 nanometers, greater than or equal to 620 nanometers, greater than or equal to 630 nanometers, greater than or equal to 640 nanometers, greater than or equal to 650 nanometers, greater than or equal to 660 nanometers, greater than or equal to 670 nanometers, greater than or equal to 680 nanometers, greater than or equal to 690 nanometers, greater than or equal to 700 nanometers, greater than or equal to 710 nanometers, greater than or equal to 720 nanometers, greater than or equal to 730 nanometers, greater than or equal to 740 nanometers, or greater than or equal to 750 nanometers.

“Biocompatibility” refers to the tolerance of a cell (e.g., prokaryotic or eukaryotic) to a coelenterazine analogue (e.g., a compound disclosed herein). Biocompatibility of a coelenterazine analogue is related to the stress it causes on the host cell.

Enhanced biocompatibility of the coelenterazine analogues (e.g., compounds disclosed herein), may be determined by measuring cell viability and/or growth rate of cells. For example, enhanced biocompatibility of the coelenterazine analogues may be determined by measuring cell viability in the absence of luciferase expression of cells exposed to the coelenterazine analogues compared to native or known coelenterazines to determine how compatible and/or toxic the coelenterazine analogues are to the cells.

In particular, enhanced biocompatibility may be determined using cell viability analysis (e.g., using the CELLTITER-GLO® Luminescent Cell Viability assay), an apoptosis assay (e.g., using the CASPASE-GLO® technology), or another method known in the art. The effect of the disclosed compounds on cell viability or apoptosis may be compared to the effect of native or known coelenterazine analogues on cell viability or apoptosis.

Enhanced biocompatibility may also be determined by measuring the effect of the coelenterazine analogues (e.g., compounds disclosed herein) on cell growth or gene expression. For example, enhanced biocompatibility of compounds disclosed herein may be determined by measuring the cell number after a period of time or by determining the expression of stress response genes in a sample of cells that are exposed to compounds disclosed herein compared to cells exposed to a native or known coelenterazine or no coelenterazine. The effect of the disclosed compounds on cell growth or gene expression may be compared to a native or known coelenterazine.

### B. Synthesis Methods

Compounds disclosed herein may be prepared by synthetic processes or by metabolic processes. Preparation of the compounds by metabolic processes includes those occurring in the human or animal body (in vivo) or processes occurring in vitro.

Compounds disclosed herein can be synthesized by a variety of methods, including those shown in the Examples. Optimum reaction conditions and reaction times for each individual step can vary depending on the particular reactants employed and substituents present in the reactants used. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step) or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. Methods of Use and Kits

The compounds of the disclosure may be used in any way that luciferase substrates, e.g., coelenterazine analogues, have been used. For example, they may be used in a bioluminogenic method that employs an analogue of coelenterazine to detect one or more molecules in a sample, e.g., an enzyme, a cofactor for an enzymatic reaction, an enzyme substrate, an enzyme inhibitor, an enzyme activator, or OH radicals, or one or more conditions, e.g., redox conditions. The sample may include an animal (e.g., a vertebrate), a plant, a fungus, physiological fluid (e.g., blood, plasma, urine, mucous secretions), a cell, a cell lysate, a cell supernatant, or a purified fraction of a cell (e.g., a subcellular fraction). The presence, amount, spectral distribution, emission kinetics, or specific activity of such a molecule may be detected or quantified. The molecule may be detected or quantified in solution, including multiphasic solutions (e.g., emulsions or suspensions), or on solid supports (e.g., particles, capillaries, or assay vessels).

In certain embodiments, the compounds disclosed herein may be used to quantify a molecule of interest. In some embodiments, a coelenterazine analogue (e.g., a native or known coelenterazine or a compound disclosed herein) can be used as a probe of a specific biochemical activity, e.g., apoptosis or drug metabolism.

In certain embodiments, the compounds disclosed herein can be used with an inhibitor of Oplophorus-derived luciferases and/or Oplophorus-luciferase derived bioluminescent complexes. Exemplary inhibitors of Oplophorus-derived luciferases and/or Oplophorus-luciferase derived bioluminescent complexes are described, for example, in International Patent Publication Nos. WO 2016/210294, WO 2018/125992, WO 2019/232384, and WO 2019/213119, each of which is herein incorporated by reference in its entirety.

In certain embodiments, the compounds disclosed herein can be used for detecting luminescence in live cells, e.g., in vivo. In some embodiments, a luciferase can be expressed in cells (as a reporter or otherwise), and the cells treated with a coelenterazine analogue (e.g., a compound disclosed herein), which will permeate cells in culture, react with the luciferase, and generate luminescence. In addition to being cell permeant, the compounds disclosed herein show comparable biocompatibility to native coelenterazine in terms of cell viability. In some embodiments, the compounds disclosed herein containing chemical modifications known to increase the stability of native coelenterazine in media can be synthesized and used for more robust, live cell luciferase-based reporter assays. In still other embodiments, a sample (including cells, tissues, animals, etc.) containing a luciferase and a compound disclosed herein may be assayed using various microscopy and imaging techniques, e.g., in vivo imaging. In still other embodiments, a secretable luciferase is expressed in cells as part of a live-cell reporter system.

In certain embodiments, the compounds disclosed herein can be used in multiplexed bioluminescence assays, such as multiplexed biochemical assays, antibody-based assays, cell-based assays, and in vivo imaging. Using red-shifted substrates, such as those disclosed herein, allows multiple colors to be obtained from the same enzyme either by switching substrates or by adding a mixture of substrates, allowing for measurement of multiple parameters simultaneously, either at a defined point in time (e.g., an end point assay) or over time with multiple measurements (e.g., a live cell assay). Such multiplexing can be in the form of a plate based assay, an imaging assay, a rapid test (such as a lateral flow strip), and many other types of assays.

In certain embodiments, the compounds disclosed herein can be used in multiplex bioluminescence assays where there are two or more bioluminescent systems with no substrate cross reactivity. In a common embodiment, one of the luciferases is an Oplophorus-derived luciferase in combination with the compounds described herein. and the second luciferase is derived from a beetle luciferase with a substrate derived from D-luciferin. These systems can be used to detect and deconvolute two biological events sequentially or simultaneously. This may include expression of two genes that express luciferase fusions, detection of protein-protein interactions, tracking two different cell types in a model organism or detecting two different antibodies, each labeled with an orthogonal luciferase, for example in a multiplex immunoassay. In some configurations, one luciferase/luciferin gives a signal that is correlated with the biological event to be measured, and the second is a control signal designed to normalize the first signal. One embodiment is the detection of T-cells infiltrating a tumor, where one luciferase is expressed in the T-cells and the second is in the tumor cells. This allows discrimination of the two cell types even when they are co-localized in a research animal such as a mouse. For a specific example of this experiment, see Su et al. (2020) Nature Methods 17: 852-860.

In certain embodiments, the compounds disclosed herein may be used as substrates within a multiplex luminescent assay. In these embodiments, there is a combination of an Oplophorus-derived luciferase that is unfused and an Oplophorus-derived luciferase that is fused to an energy acceptor, or two or more Oplophorus-derived luciferases that are fused to spectrally distinguishable energy acceptors. In some embodiments, these luciferases may be expressed by different genes to discriminate them, be in different cells in an animal for the purposes of distinguishing the cells by color, or fused to antibodies or antibody mimetics for multiplex immunoassays. In all of these cases, a single substrate is acted upon by all of these luciferases simultaneously, and the relative activities of the different luciferases may be deconvoluted through the color of the light emitted from the associated energy acceptor(s). As an example, antibodies that bind two different markers can be distinguished by labeling one antibody with unfused luciferase and a second with fused luciferase and quantitating both the light emission and the color of the light emitted.

In certain embodiments, the compounds disclosed herein may be provided as part of a kit. In some embodiments, the kit may include one or more luciferases (in the form of a polypeptide, a polynucleotide, or both) and a coelenterazine analogue disclosed herein, along with suitable reagents and instructions to enable a user to perform assays such as those disclosed herein. The kit may also include one or more buffers such as those disclosed herein. In some embodiments, the kit may further include an inhibitor of Oplophorus-derived luciferases and/or Oplophorus-luciferase derived bioluminescent complexes, as described above.

Buffers include citric acid or citrate buffer, MES, 1,4-Piperazinediethanesulfonic acid, or HEPES; inorganic phosphate, for example, in the form pyrophosphate or potassium phosphate; a chelator such as EDTA, CDTA or 1,2-Diaminocyclohexanetetraacetic acid; a salt such as sodium fluoride, magnesium sulfate; a surfactant or detergent such as TERGITOL® (e.g. a non-ionic nonylphenol ethoxylate), dodecyltrimethylammonium bromide (DTAB) or THESIT® (hydroxypolyethoxydodecane); a defoamer such as INDUSTROL® DF204 (organic defoamer) or MAZU® DF (silicone defoamer); a protein stabilizer such as gelatin, PRIONEX® 10% (gelatin, Type A) or albumin (e.g. BSA, HSA) or glycerol; adenosine triphosphate (ATP) or adenosine monophosphate (AMP). Other components may include polyethylene glycol, polyvinyl pyridine, crown ether, or cyclodextrin.

### A. In Vivo Imaging

The compounds of the disclosure can be used for imaging of live cells such as in vivo and ex vivo bioluminescence imaging. For example, the compounds of the disclosure can be used with a coelenterazine utilizing luciferase for bioluminescence imaging tissue sections or cells in a live animal. In vivo bioluminescence imaging is a versatile and sensitive tool based on the detection of emitted light from cells or tissues. Bioluminescence has been used to track tumor cells, bacterial and viral infections, gene expression and treatment response in a non-invasive manner. Bioluminescence imaging provides longitudinal monitoring of a disease course in the same animal, a desirable alternative to analyzing a number of animals at many time points during the course of the disease. In some embodiments, the compounds of the disclosure can be used in vivo to monitor biological processes such as cell movement, tumor progression, gene expression, and viral infection in a variety of animal models. In some embodiments, the compounds of the disclosure can be used for imaging in a transgenic animal, such as a transgenic mouse. Transgenic animals, including cells or tissues, can represent models of cell function and disease in humans. Accordingly, these animals are useful in studying the mechanisms behind cell function and related events, in generating and testing products (e.g., antibodies, small molecules etc.), and in treating and diagnosing associated human diseases, including cancer and autoimmune conditions. In some embodiments, the transgenic animal can further provide an indication of the safety of a particular agent for administration to a human. The effect of the agent can be studied by administration of a particular agent and the compounds of the disclosure to specific cells or the whole body and performing bioluminescent imaging to look for specific effects. The animal- and cell-based models and compounds of the disclosure may be used to identify drugs, pharmaceuticals, therapies, and interventions that may be effective in treating disease.

In some embodiments, the compounds of the disclosure can be used for bioluminescence imaging of cells or animals that have been transformed to express a fusion protein, such as a fusion protein comprising a luciferase. In some embodiments, the transgenic animal or cell can express a fusion protein comprising a luciferase. In some embodiments, the luciferase can be a coelenterazine-utilizing luciferase, such as an Oplophorus or Oplophorus-derived luciferase, a Renilla luciferase, a Gaussia luciferase, such as a Gaussia princeps luciferase, a Metridia luciferase, such as Metridia longa and Metridia pacifica luciferases, a Vargula luciferase, such as a Vargula hilgendorfii luciferase, a Pleuromamma xiphias luciferase, and variants, recombinants, and mutants thereof. In some embodiments, the polynucleotide sequence encoding the fusion protein is operably linked to a promoter. In some embodiments, the promoter can be a constitutive promoter, an inducible promoter, a repressible promoter, or a regulatable promoter. In some embodiments, the promoter can also be a tissue specific promoter.

In some embodiments, a fusion protein of a bioluminescent protein and a heterologous protein of interest, such as a fluorescent protein, may be connected directly to each other by peptide bonds or may be separated by intervening amino acid sequences. In some embodiments, the fusion polypeptides may also contain sequences exogenous to the bioluminescent protein and the heterologous protein of interest, such as a fluorescent protein. For example, the fusion protein may include targeting or localization sequences, tag sequences, sequences of other fluorescent proteins or bioluminescent proteins, or other chromophores. In some embodiments, the targeting sequence may direct localization of the fusion protein to a specific tissue, cell-type (e.g., muscle, heart, or neural cell), cellular compartment (e.g., mitochondria or other organelle, nucleus, cytoplasm, or plasma membrane), or protein. Moreover, the fusion may contain sequences from multiple fluorescent or bioluminescent proteins, or variants thereof, and/or other selected proteins. In some embodiments, the luciferase is fused to a HALOTAG® protein or a fluorescent protein, such as green fluorescent protein (GFP), red fluorescent protein (RFP), or orange-red fluorescent protein.

The bioluminescence produced within a cell, such as in a cell of a transgenic animal, is capable of being imaged or detected by a variety of means well known in the art. For example, the fusion protein and the compounds of the disclosure that have localized to their intended sites in a transgenic animal may be imaged in a number of ways. A reasonable estimate of the time to achieve localization may be made by one skilled in the art. Furthermore, the state of localization as a function of time may be followed by imaging the bioluminescence generated from the fusion protein and the compounds of the disclosure. Since the imaging, or measuring photon emission from the subject, may last up to tens of minutes, the transgenic animal can be immobilized during the imaging process.

In vivo imaging can be performed using the naked eye or any sort for camera (still or video). Imaging bioluminescence involves the use of, e.g., a photodetector capable of detecting extremely low levels of light—typically single photon events—and integrating photon emission until an image can be constructed. Examples of such sensitive photodetectors include devices that intensify the single photon events before the events are detected by a camera, and cameras (cooled, for example, with liquid nitrogen) that are capable of detecting single photons over the background noise inherent in a detection system. The “photodetector device” used should have a high enough sensitivity to enable the imaging of faint light from within a mammal in a reasonable amount of time, and to use the signal from such a device to construct an image.

The bioluminescence signal can be detected with a highly sensitive, intensified charge coupled device (CCD) camera. In certain embodiments, an intensified CCD camera sensitive enough to detect a bioluminescent signal and with wide enough dynamic range to also detect a fluorescent signal is used for imaging. Suitable cameras are known in the art and include, but are not limited to, an Olympus LV200 Bioluminescence Imaging System, an integrated imaging system (IVIS™ Imaging System, Caliper Life Sciences) controlled using LivingImage™ software (Caliper Life Sciences), or a custom-built two-photon fluorescence lifetime imaging microscope (Yasuda Curr Opin Neurobiol. 2006; 16:551-561). In some embodiments, the camera is mounted in a light-proof container that provides anesthesia, platforms for the animal, such as a mouse, and internal lighting.

The in vivo imaging can be a non-invasive whole animal imaging that have been described (Contag, C., U.S. Pat. No. 5,650,135, Jul. 22, 1997), herein incorporated by reference; Contag, P., et al, Nature Medicine 4(2):245-247, 1998; Contag, C., et al, OSA TOPS on Biomedical Optical Spectroscopy and Diagnostics 3:220-224, 1996; Contag, C. H., Photochemistry and Photobiology 66(4):523-531, 1997; Contag, C. H., al, Molecular Microbiology 18(4):593-603, 1995). Sensitivity of detecting light emitted from internal organs depends on several factors, including the level of luciferase expression, the depth of labeled cells within the body (the distance that the photons must travel through tissue), and the sensitivity of the detection system.

“Photon amplification devices” amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Image processors process signals generated by photodetector devices which count photons in order to construct an image which can be, for example, displayed on a monitor or printed on a video printer. Such image processors are typically sold as part of systems which include the sensitive photon-counting cameras described above, and accordingly, are available from the same sources. The image processors are usually connected to a personal computer, such as an IBM-compatible PC or an Apple Macintosh (Apple Computer, Cupertino, Calif), which may or may not be included as part of a purchased imaging system. Once the images are in the form of digital files, they can be manipulated by a variety of image processing programs (such as “ADOBE PHOTOSHOP,” Adobe Systems, Adobe Systems, Mt. View, Calif) and printed.

It will be understood that the entire animal or subject need not necessarily be in the detection field of the photodetection device. For example, if one is measuring a fusion protein targeted to a particular region of the subject, only light from that region, and a sufficient surrounding “dark” zone, need be measured to obtain the desired information.

Once a photon emission image is generated, it is typically superimposed on a “normal” reflected light image of the subject to provide a frame of reference for the source of the emitted photons (i.e., localize the fusion proteins with respect to the subject). A “composite” image formed by the superimposition of the photon emission image on the reflected light image is then analyzed to determine the location and/or amount of a target in the subject.

### B. Bioluminescence Resonance Energy Transfer (BRET)

The disclosed compounds can be used in any method for detecting ligand-protein and/or protein-protein interactions. In some embodiments, the compounds of the disclosure can be used in an in vivo or in vitro bioluminescence resonance energy transfer (BRET) system. With respect to BRET, energy transfer from a bioluminescent donor to a fluorescent acceptor results in a shift in the spectral distribution of the emission of light. This energy transfer may enable real-time monitoring of protein-protein or ligand-protein interaction in vitro or in vivo, such as the interaction and dissociation of the partners. Examples of BRET systems, such as NanoBRET™ systems, are described, for example, in U.S. Pat. No. 10,024,862, U.S. Patent Publication No. 2014/0194307, U.S. Pat. No. 10,067,149, and U.S. Patent Publication No. 2014/0194325.

In BRET assays, as the magnitude of energy transfer is proportional to the overlap of the emission spectrum of the donor and the excitation spectrum of the acceptor according to the Forster equation, red-shifting the emission through use of red-shifted substrates disclosed herein allows better energy transfer to red, far-red, and infrared acceptors. This is advantageous for further multiplexing as well as tissue penetration of the emitted light.

In some embodiments, the luminescent enzymes used in BRET analysis can be used to determine if two molecules are capable of binding to each other or co-localize in a cell. For example, a luminescent enzyme can be used as a bioluminescence donor molecule which is combined with a molecule or protein of interest to create a first fusion protein. In some embodiments, the luminescent enzyme can be conjugated with an antibody, a protein, a receptor, a drug, a drug carrier, a peptide, a sugar, a fatty acid, a nanoparticle, or other biomolecule. In various embodiments, the first fusion protein contains a luminescent enzyme and a protein of interest. In various embodiments, the first fusion proteins containing the luminescent enzyme can be used in BRET analysis to detect protein/protein interaction in systems including but not limited to cell lysates, intact cells, and living animals. In some embodiments, the BRET analysis can also include an inhibitor of Oplophorus-derived luciferases and/or Oplophorus-luciferase derived bioluminescent complexes, as described above.

In some embodiments, the fluorescent acceptor can be a fluorophore, such as a fluorescent protein, fluorescent molecule, fluorescent label, or fluorescent tracer. In some embodiments, the fluorescent tracer can be a small molecule tagged to a fluorophore. In some embodiments, the fluorescent acceptor can be a second fusion protein that includes a fluorescent acceptor conjugated to an antibody, a protein, a receptor, a drug, a drug carrier, a peptide, a sugar, a fatty acid, a nanoparticle, or other biomolecule.

In various embodiments, HALOTAG® can be used as a fluorescent acceptor molecule. In some embodiments, HALOTAG® or can be fused to a second protein of interest or to a luminescent enzyme. For example, a luminescent enzyme can be fused to HALOTAG®, expressed in cells or animals, and labeled with a fluorescent HALOTAG® ligand such as HALOTAG® TMR ligand. In another example, a luminescent enzyme can be fused to fluorescent protein and expressed in cells or animals. In some embodiments, BRET may be performed using luminescent enzymes in combination with fluorescent proteins, including but not limited to GFP, RFP, orange-red fluorescent protein, or fluorescent labels including fluorescein, rhodamine green, Oregon green, or Alexa 488, to name a few non-limiting examples.

In some embodiments, the disclosed compounds can be used in a target engagement assay, such as NANOBRET™ Target Engagement (TE) Assay, to measure compound binding at select target proteins, such as drug:target interaction, in intact cells in real time. For example, the NANOBRET™ TE Assay can include four components: an expressed cellular target protein that is fused to the bright NANOLUC® luciferase; a cell-permeable fluorescent tracer that specifically binds to the target protein; one or more of the disclosed compounds used as a substrate for the NANOLUC® luciferase; and a cell-impermeable inhibitor for NANOLUC® luciferase. The assay uses bioluminescence resonance energy transfer (BRET), achieved by transferring the luminescent energy from NANOLUC® luciferase to the fluorescent tracer that is bound to the target protein-NANOLUC® fusion. This energy transfer makes it possible to directly measure compound binding affinity as well as compound-target residence time.

In some embodiments, compounds that are applied to the cells and specifically can engage the intracellular target protein-NANOLUC® fusion and will result in a decrease in BRET. In some embodiments, to ensure accurate assessment of intracellular target engagement, a NANOLUC® inhibitor can be used to mitigate any extracellular NANOLUC® signal that may arise from cells compromised during handling, while not adversely affecting NANOLUC® luciferase expressed within healthy living cells.

The BRET system may further comprise a photodetector or imaging device for detecting light emitted from the bioluminescent fusion protein, such as, but not limited to, an optical microscope, a digital microscope, a luminometer, a charged coupled device (CCD) image sensor, a complementary metal-oxide-semiconductor (CMOS) image sensor, or a digital camera.

### C. Formulation and Administration

For whole animal studies, the disclosed imaging probes are preferably formulated for parenteral administration. Parenteral formulations can be prepared as aqueous compositions using techniques known in the art. Typically, such compositions are prepared as solutions or suspensions; solid forms suitable to prepare solutions or suspensions upon the addition of a reconstitution medium; emulsions, such as water-in-oil (w/o) emulsions, oil-in-water (o/w) emulsions, and microemulsions thereof, liposomes, or emulsomes.

As used herein, the term “parenteral” refers to modes of administration which include intravenous, intramuscular, intraperitoneal, intrasternal, subcutaneous and intraarticular injection and infusion.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric, or nonionic surface active agents. Suitable anionic surfactants include, but are not limited to, those containing carboxylate, sulfonate, and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### 4. Examples

### Example 1. Synthesis of Intermediates

### Synthesis of Intermediate 1: 3-bromo-5-iodopyrazin-2-amine

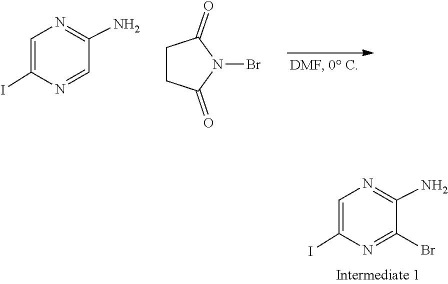 [SMILES: Nc1cnc(I)cn1 ; O=C1CCC(=O)N1Br ; Nc1ncc(I)nc1Br]

To a 250 mL flask, 5-iodopyrazin-2-amine (15.5 g, 70.1 mmol) and DMF (150 mL) were added. The mixture was stirred and cooled at 0° C. To the mixture, the NBS (14.4 g, 80.7 mmol) was added. The mixture was allowed to warm to RT and stirred for 14 h. The mixture was diluted in water (400 mL), saturated NaHSO₃ (20 mL), and EtOAc (400 mL). The organic layer was dried, filtered, and concentrated onto Celite. The mixture was purified by silica gel chromatography with 0-30% EtOAc in heptane as eluent. During concentration, the solid crystallized and was collected by filtration to Intermediate 1. LCMS (C₄H₃BrIN₃) (ES, m/z) 300 [M+H]⁺.

### Synthesis of Intermediate 2: 3-bromo-5-phenylpyrazin-2-amine

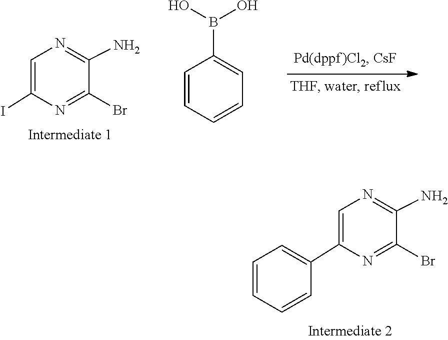 [SMILES: Nc1ncc(I)nc1Br ; OB(O)c1ccccc1 ; Nc1ncc(-c2ccccc2)nc1Br]

To a 100 mL flask, Intermediate 1 (3.20 g, 10.7 mmol), Pd(dppf)Cl₂ (156 mg, 0.213 mmol), phenyl boronic acid (1.43 g, 11.7 mmol), cesium fluoride (3.24 g, 21.3 mmol), THF (20 mL), and water (2 mL) were added. The mixture was sparged with nitrogen for 2 min. The mixture was stirred and heated at reflux for 4 h. The mixture was cooled to RT, adsorbed to Celite, and purified by silica gel chromatography with 0-50% EtOAc in heptane as eluent to afford Intermediate 2. LCMS (C₁₀H₈BrN₃) (ES, m/z) 250 [M+H]⁺.

### Synthesis of Intermediate 3: Tert-butyl 2-((3-bromo-5-iodopyrazin-2-yl)amino)-2-(diethoxyphosphoryl)acetate

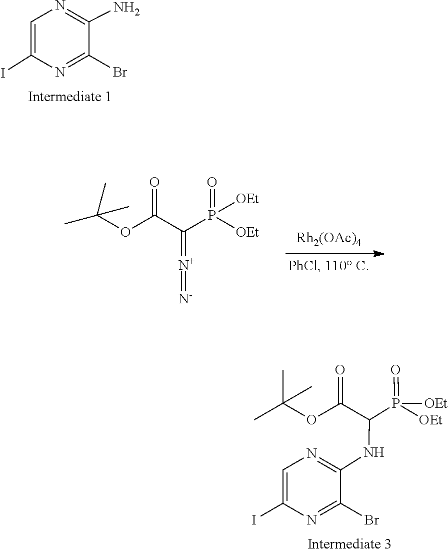 [SMILES: Nc1ncc(I)nc1Br ; CCOP(=O)(OCC)C(=[N+]=[N-])C(=O)OC(C)(C)C ; CCOP(=O)(OCC)C(Nc1ncc(I)nc1Br)C(=O)OC(C)(C)C ; "Rh2(OAc)4"]

To a 250 mL flask, the Intermediate 1 (7.00 g, 23.3 mmol), Rh₂(OAc)₂ (478 mg, 1.17 mmol), chlorobenzene (35 mL), and tert-butyl 2-diazo-2-(diethoxyphosphoryl)acetate (7.60 g, 27.3 mmol) were added. The mixture was stirred and heated at 110° C. for 18 h. The mixture was cooled to RT and loaded directly onto a silica gel column and purified with 0-100% EtOAc in heptane as eluent to afford Intermediate 3. LCMS (C₁₄H₂₂BrIN₃O₅P) (ES, m/z) 550 [M+H]⁺.

### Synthesis of Intermediate 3: Tert-butyl 2-((3-bromo-5-iodopyrazin-2-yl)amino)-2-(diethoxyphosphoryl)acetate

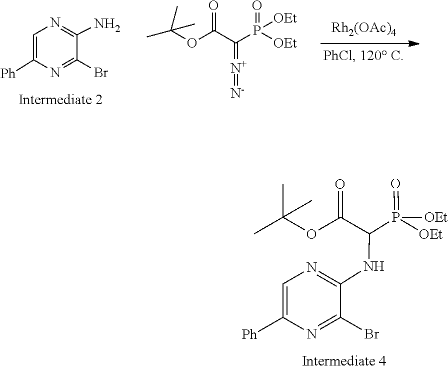 [SMILES: Nc1ncc(-c2ccccc2)nc1Br ; CCOP(=O)(OCC)C(=[N+]=[N-])C(=O)OC(C)(C)C ; CCOP(=O)(OCC)C(Nc1ncc(-c2ccccc2)nc1Br)C(=O)OC(C)(C)C ; "Rh2(OAc)4"]

To a 250 mL flask, the Intermediate 2 (500 mg, 2.00 mmol), Rh₂(OAc)₂ (41.0 mg, 0.100 mmol), chlorobenzene (3 mL), and tert-butyl 2-diazo-2-(diethoxyphosphoryl)acetate (612 mg, 2.20 mmol) were added. The mixture was stirred and heated at 120° C. for 18 h. The mixture was cooled to RT and loaded directly onto a silica gel column and purified with 0-100% EtOAc in heptane as eluent to afford Intermediate 4. LCMS (C₂₀H₂₇BrN₃O₅P) (ES, m/z) 500 [M+H]⁺.

The intermediate in the following table was prepared analogously to Intermediate 4 from Intermediate 1.

| | | LCMS |
|---|---|---|
| Inter- | | (ES, m/z) |
| mediate | Structure | [M + H]⁺ |
| 5 | 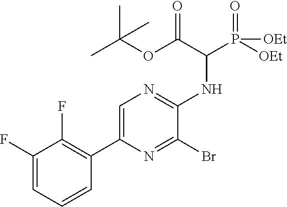 [SMILES: CCOP(=O)(OCC)C(Nc1ncc(-c2cccc(F)c2F)nc1Br)C(=O)OC(C)(C)C] | 536 |

### Synthesis of Intermediate 6: Tert-butyl (Z)-2-((3-bromo-5-iodopyrazin-2-yl)amino)-3-(furan-2-yl)acrylate

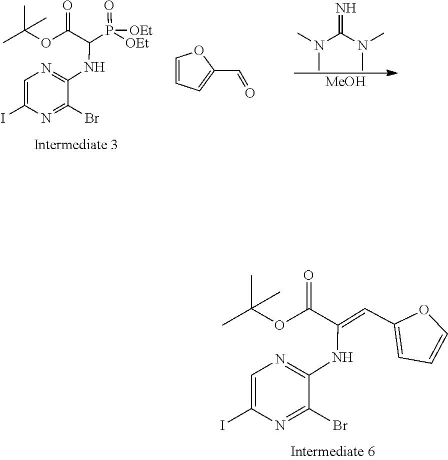

To a 250 mL flask, the Intermediate 3 (6.49 g, 11.8 mmol), furfural (1.13 mL, 13.0 mmol), and methanol (50 mL) were added. To the stirring mixture, 1,1,3,3-tetramethylguanidine (3.58 mL, 29.5 mmol) was added, dropwise. The mixture was stirred for 10 min. The mixture was adsorbed to Celite and purified by silica gel chromatography with 0-50% EtOAc in heptane as eluent to afford Intermediate 6. LCMS (C₁₅H₁₅BrIN₃O₃) (ES, m/z) 492 [M+H]⁺.

### Synthesis of Intermediate 7: Tert-butyl (Z)-2-((3-bromo-5-phenylpyrazin-2-yl)amino)-3-(furan-2-yl)acrylate

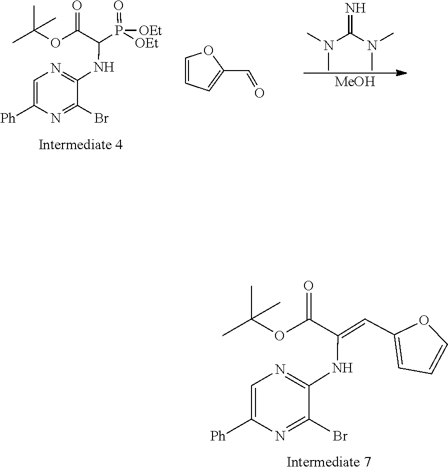

To a 20 mL vial, the Intermediate 4 (510 mg, 1.02 mmol), furfural (0.098 mL, 1.12 mmol), and methanol (3 mL) were added. To the stirring mixture, 1,1,3,3-tetramethylguanidine (0.316 mL, 2.55 mmol) was added, dropwise. The mixture was stirred for 10 min. The mixture was adsorbed to Celite and purified by silica gel chromatography with 0-35% EtOAc in heptane as eluent to afford Intermediate 7. LCMS (C₂₁H₂₀BrN₃O₃) (ES, m/z) 442 [M+H]⁺.

### Synthesis of Intermediate 8: 3-Chloro-5-(4,4,5,5-tetramethyl-1,3,2-dioxaborolan-2-yl)pyridazine

### Step 1: 5-Bromo-3-chloropyridazine

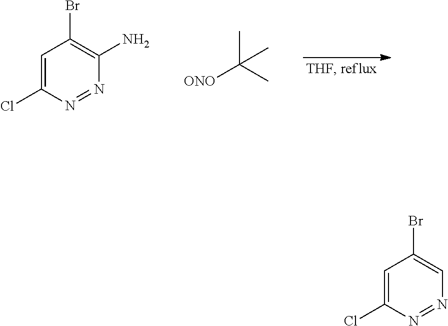

To a 100 mL flask, 4-bromo-6-chloropyridazin-3-amine (3.00 g, 14.4 mmol), THF (20 mL), and tert-butyl nitrite (2.91 mL, 24.5 mmol) were added. The mixture was stirred and heated at reflux for 1 h. The mixture was cooled to RT, adsorbed to Celite, and purified by silica gel chromatography with 0-40% EtOAc in heptane as eluent to afford 5-bromo-3-chloropyridazine. LCMS (C₄H₂BrClN₂) (ES, m/z) 193 [M+H]⁺.

### Step 2: 3-Chloro-5-(4,4,5,5-tetramethyl-1,3,2-dioxaborolan-2-yl)pyridazine

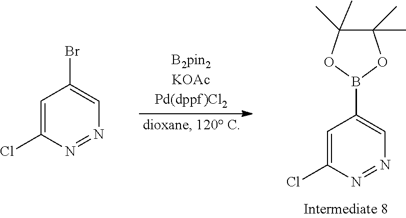 [SMILES: Clc1cc(Br)cnn1 ; CC1(C)OB(c2cnnc(Cl)c2)OC1(C)C]

To a 20 mL vial, 5-bromo-3-chloropyridazine (300 mg, 1.55 mmol), Pd(dppf)Cl₂ (56.7 mg, 0.0776 mmol), B₂pin₂ (433 mg, 1.71 mmol), KOAc (304 mg, 3.10 mmol), and dioxane (5 mL) were added. The mixture was degassed with nitrogen for 1 min. The mixture was stirred and heated at 120° C. for 14 h. The mixture was cooled to RT. The mixture was diluted in EtOAc and filtered through Celite. The solvents of the filtrate were evaporated, and the residue was coevaporated with toluene two times. To the residue, Et₂O (10 mL) was added. The mixture was stirred for 2 h. The mixture was filtered through Celite, and the filtrate was concentrated. To the residue was added 10 mL of heptane. The mixture was stirred for 14 h. An oil came out that stuck to the sides of the vial. The supernatant was decanted and saved. The oily residue was again treated with heptane (10 mL), stirring for 2 h. The supernatant was decanted again. The combined supernatants were filtered, and the filtrate was concentrated to afford Intermediate 8. LCMS (observed hydrolysis to the boronic acid) (C₄H₄BClN₂O₂) (ES, m/z) 159 [M+H]⁺.

### Synthesis of Intermediate 9: Tert-butyl 2-((3-bromo-5-iodopyrazin-2-yl)amino)-3-(furan-2-yl)propanoate

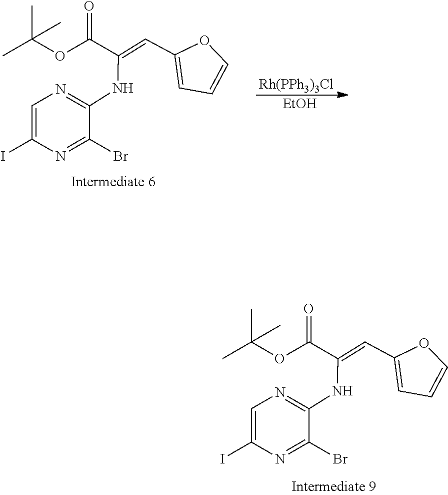 [SMILES: "EtOH" ; CC(C)(C)OC(=O)/C(=C/c1ccco1)Nc1ncc(I)nc1Br ; CC(C)(C)OC(=O)/C(=C/c1ccco1)Nc1ncc(I)nc1Br ; "Rh(PPh3)3Cl"]

To a 250 mL flask, the Intermediate 6 (2.81 g, 5.71 mmol), Wilkinson's catalyst (1.06 g, 1.14 mmol), and EtOH (125 mL) were added. The mixture was degassed and placed under an H₂ atmosphere from a balloon. After 6 days, the mixture was concentrated, and the residue was purified by silica gel chromatography with 0-30% EtOAc in heptane to afford tert-butyl 2-((3-bromo-5-iodopyrazin-2-yl)amino)-3-(furan-2-yl)propanoate Intermediate 9. LCMS (C₁₅H₁₇BrIN₃O₃) (ES, m/z) 438 [M+H−C₄H₈]⁺.

The intermediates in the following table were prepared analogously to Intermediate 9 from Intermediate 4 or Intermediate 5 and the corresponding aldehydes.

| Intermediate | Structure | LCMS (ES, m/z) [M + H]⁺ |
|---|---|---|
| 10 | 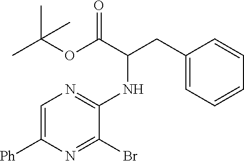 [SMILES: CC(C)(C)OC(=O)C(Cc1ccccc1)Nc1ncc(-c2ccccc2)nc1Br] | 454 |
| 11 | 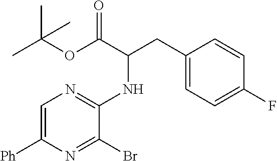 [SMILES: CC(C)(C)OC(=O)C(Cc1ccc(F)cc1)Nc1ncc(-c2ccccc2)nc1Br] | 472 |
| 12 | 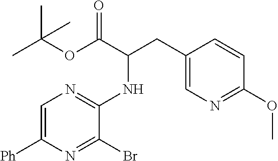 [SMILES: COc1ccc(CC(Nc2ncc(-c3ccccc3)nc2Br)C(=O)OC(C)(C)C)cn1] | 485 |
| 13 | 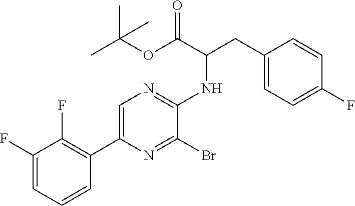 [SMILES: CC(C)(C)OC(=O)C(Cc1ccc(F)cc1)Nc1ncc(-c2cccc(F)c2F)nc1Br] | 508 |

### Example 2. Synthesis of Compounds

### Synthesis of Compound 9894: 8-(6-Chloropyridazin-4-yl)-2-(furan-2-ylmethyl)-6-phenylimidazo[1,2-a]pyrazin-3(7H)-one

### Step 1: Tert-butyl (Z)-2-((3-(6-chloropyridazin-4-yl)-5-phenylpyrazin-2-yl)amino)-3-(furan-2-yl)acrylate

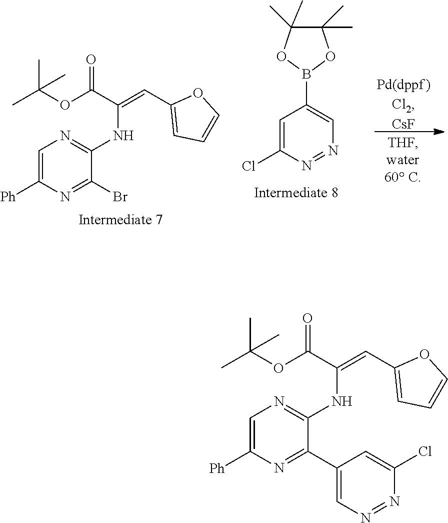 [SMILES: CC(C)(C)OC(=O)/C(=C/c1ccco1)Nc1ncc(-c2ccccc2)nc1Br ; CC1(C)OB(c2cnnc(Cl)c2)OC1(C)C ; "Pd(dppf)Cl2,CsF" ; CC(C)(C)OC(=O)/C(=C/c1ccco1)Nc1ncc(-c2ccccc2)nc1-c1cnnc(Cl)c1]

To a 20 mL vial, Intermediate 7 (61.0 mg, 0.138 mmol), Intermediate 8 (66.3 mg, 0.276 mmol), CsF (105 mg, 0.690 mmol), Pd(dppf)Cl₂ (5.1 mg, 0.0069 mmol), THF (2 mL), and water (0.3 mL) were added. The mixture was sparged with nitrogen for 2 min. The mixture was stirred and heated at 60° C. After 14 h, the mixture was adsorbed to Celite and purified by silica gel chromatography with 0-60% EtOAc in heptane as eluent to afford tert-butyl (Z)-2-((3-(6-chloropyridazin-4-yl)-5-phenylpyrazin-2-yl)amino)-3-(furan-2-yl)acrylate. LCMS (C₂₅H₂₂ClN₅O₃) (ES, m/z) 476 [M+H]⁺.

### Step 2: (Z)-2-((3-(6-chloropyridazin-4-yl)-5-phenylpyrazin-2-yl)amino)-3-(furan-2-yl)acrylic acid

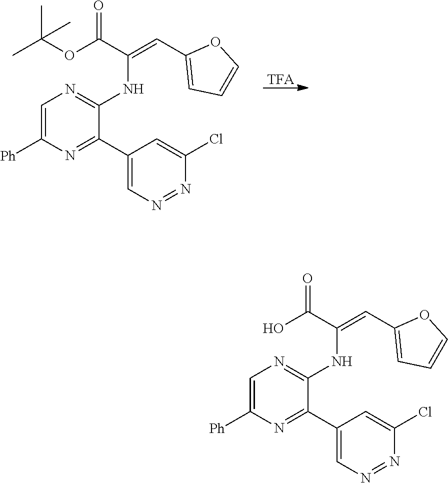 [SMILES: CC(C)(C)OC(=O)/C(=C/c1ccco1)Nc1ncc(-c2ccccc2)nc1-c1cnnc(Cl)c1 ; O=C(O)/C(=C/c1ccco1)Nc1ncc(-c2ccccc2)nc1-c1cnnc(Cl)c1 ; "TFA"]

To a 20 mL vial, tert-butyl (Z)-2-((3-(6-chloropyridazin-4-yl)-5-phenylpyrazin-2-yl)amino)-3-(furan-2-yl)acrylate (24.0 mg, 0.0504 mmol) and TFA (1 mL) were added. The mixture was stirred for 1 h. The solvents were evaporated to afford (Z)-2-((3-(6-chloropyridazin-4-yl)-5-phenylpyrazin-2-yl)amino)-3-(furan-2-yl)acrylic acid. LCMS (C₂₁H₁₄ClN₅O₃) (ES, m/z) 420 [M+H]⁺.

### Step 3: (Z)-8-(6-chloropyridazin-4-yl)-2-(furan-2-ylmethylene)-6-phenylimidazo[1,2-a]pyrazin-3(2H)-one

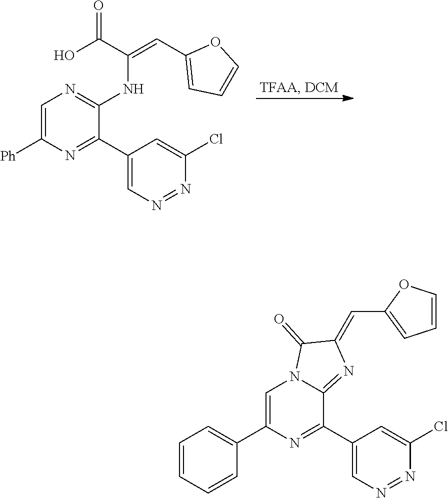

To a 20 mL vial, (Z)-2-((3-(6-chloropyridazin-4-yl)-5-phenylpyrazin-2-yl)amino)-3-(furan-2-yl)acrylic acid (21.2 mg, 0.0505 mmol), DCM (1 mL), and TFAA (0.5 mL) were added. The mixture was stirred for 10 min. The solvents were evaporated to afford (Z)-8-(6-chloropyridazin-4-yl)-2-(furan-2-ylmethylene)-6-phenylimidazo[1,2-a]pyrazin-3(2H)-one. LCMS (C₂₁H₁₂ClN₅O₂) (ES, m/z) 402 [M+H]⁺.

### Step 4: 8-(6-Chloropyridazin-4-yl)-2-(furan-2-ylmethyl)-6-phenylimidazo[1,2-a]pyrazin-3(7H)-one

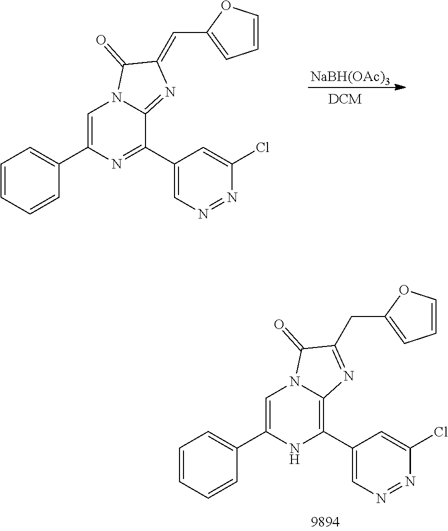 [SMILES: O=C1/C(=C/c2ccco2)N=C2C(c3cnnc(Cl)c3)=NC(c3ccccc3)=CN12 ; O=c1c(Cc2ccco2)nc2c(-c3cnnc(Cl)c3)[nH]c(-c3ccccc3)cn1-2 ; "NaBH(OAc)3"]

To a 20 mL vial, (Z)-8-(6-chloropyridazin-4-yl)-2-(furan-2-ylmethylene)-6-phenylimidazo[1,2-a]pyrazin-3(2H)-one (7.1 mg 0.0177 mmol) and DCM (2 mL) were added. The mixture was degassed with nitrogen for 1 min. To the mixture, sodium triacetoxy borohydride (37.5 mg, 0.177 mmol) was added. The mixture was stirred and heated at 50° C. for 3 h. The mixture was cooled to RT, quenched with AcOH (0.2 mL), filtered, and the solvents of the filtrate were evaporated. The residue was purified by reversed phase HPLC (MeCN/water w/0.1% TFA) to afford 9894. LCMS (C₂₁H₁₂ClN₅O₂) (ES, m/z) 404 [M+H]⁺. ¹H NMR (400 MHz, DMSO-d₆) δ 11.67 (s, 1H), 10.52 (d, J=1.7 Hz, 1H), 9.17 (d, J=1.7 Hz, 1H), 8.78 (s, 1H), 8.26 (d, J=7.7 Hz, 2H), 7.64-7.48 (m, 3H), 7.46 (t, J=7.3 Hz, 1H), 6.53 (s, 1H), 6.39 (t, J=2.6 Hz, 1H), 6.18 (d, J=3.2 Hz, 1H), 4.25 (s, 2H).

### Synthesis of Compound 10158: 8-(6-chloropyridazin-4-yl)-2-(furan-2-ylmethyl)-6-(3-hydroxyphenyl)imidazo[1,2-a]pyrazin-3(7H)-one

### Step 1: Tert-butyl (Z)-2-((3-bromo-5-(3-hydroxyphenyl)pyrazin-2-yl)amino)-3-(furan-2-yl)acrylate

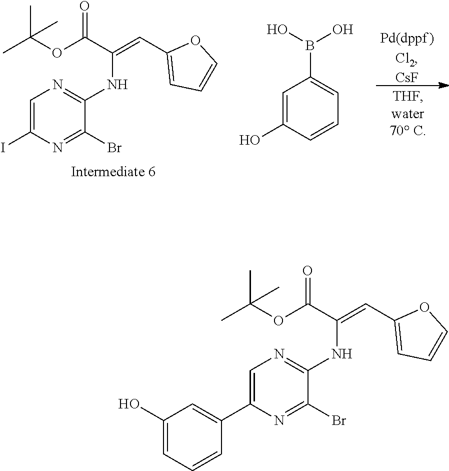 [SMILES: CC(C)(C)OC(=O)/C(=C/c1ccco1)Nc1ncc(I)nc1Br ; OB(O)c1cccc(O)c1 ; "Pd(dppf)Cl2,CsF" ; CC(C)(C)OC(=O)/C(=C/c1ccco1)Nc1ncc(-c2cccc(O)c2)nc1Br]

To a 20 mL vial, Intermediate 6 (74.0 mg, 0.150 mmol), (3-hydroxyphenyl)boronic acid (31.1 mg, 0.226 mmol), CsF (114 mg, 0.752 mmol), Pd(dppf)Cl₂ (11.0 mg, 0.0150 mmol), THF (8 mL), and water (0.8 mL) were added. The mixture was sparged with nitrogen for 2 min. The mixture was stirred and heated at 70° C. After 72 h, the mixture was adsorbed to Celite and purified by silica gel chromatography with 0-50% EtOAc in heptane as eluent to afford tert-butyl (Z)-2-((3-bromo-5-(3-hydroxyphenyl)pyrazin-2-yl)amino)-3-(furan-2-yl)acrylate. LCMS (C₂₁H₂₀BrN₃O₄) (ES, m/z) 458 [M+H]⁺.

### Step 2: Tert-butyl (Z)-2-((3-(6-chloropyridazin-4-yl)-5-(3-hydroxyphenyl)pyrazin-2-yl)amino)-3-(furan-2-yl)acrylate

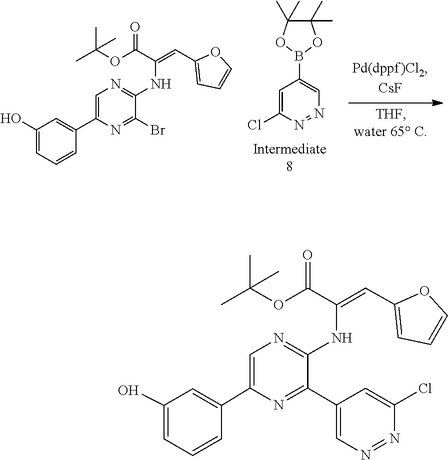 [SMILES: CC(C)(C)OC(=O)/C(=C/c1ccco1)Nc1ncc(-c2cccc(O)c2)nc1Br ; CC1(C)OB(c2cnnc(Cl)c2)OC1(C)C ; CC(C)(C)OC(=O)/C(=C/c1ccco1)Nc1ncc(-c2cccc(O)c2)nc1-c1cnnc(Cl)c1]

To a 20 mL vial, tert-butyl (Z)-2-((3-bromo-5-(3-hydroxyphenyl)pyrazin-2-yl)amino)-3-(furan-2-yl)acrylate (22.0 mg, 0.0480 mmol), Intermediate 8 (46.2 mg, 0.192 mmol), CsF (36.5 mg, 0.240 mmol), Pd(dppf)Cl₂ (3.5 mg, 0.0048 mmol), THF (1 mL), and water (0.2 mL) were added. The mixture was sparged with nitrogen for 2 min. The mixture was stirred and heated at 65° C. After 2 h, the mixture was adsorbed to Celite and purified by silica gel chromatography with 0-70% EtOAc in heptane as eluent to afford tert-butyl (Z)-2-((3-(6-chloropyridazin-4-yl)-5-(3-hydroxyphenyl)pyrazin-2-yl)amino)-3-(furan-2-yl)acrylate. LCMS (C₂₅H₂₂ClN₅O₄) (ES, m/z) 492 [M+H]⁺.

### Step 3: (Z)-2-((3-(6-chloropyridazin-4-yl)-5-(3-hydroxyphenyl)pyrazin-2-yl)amino)-3-(furan-2-yl)acrylic acid

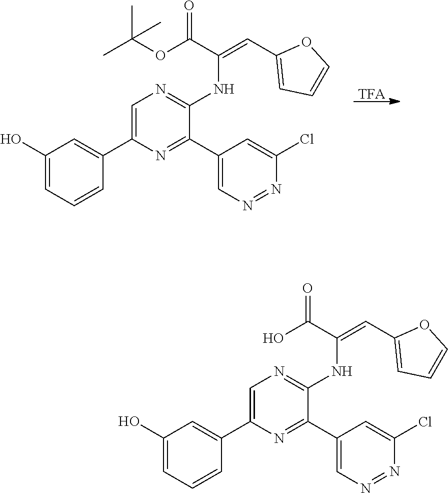 [SMILES: CC(C)(C)OC(=O)/C(=C/c1ccco1)Nc1ncc(-c2cccc(O)c2)nc1-c1cnnc(Cl)c1 ; O=C(O)/C(=C/c1ccco1)Nc1ncc(-c2cccc(O)c2)nc1-c1cnnc(Cl)c1 ; "TFA"]

To a 20 mL vial, tert-butyl (Z)-2-((3-(6-chloropyridazin-4-yl)-5-(3-hydroxyphenyl)pyrazin-2-yl)amino)-3-(furan-2-yl)acrylate (4.0 mg, 0.0081 mmol) and TFA (1 mL) were added. The mixture was stirred for 1 h. The solvents were evaporated to afford (Z)-2-((3-(6-chloropyridazin-4-yl)-5-(3-hydroxyphenyl)pyrazin-2-yl)amino)-3-(furan-2-yl)acrylic acid. LCMS (C₂₁H₁₄ClN₅O₄) (ES, m/z) 436 [M+H]⁺.

### Step 4: (Z)-8-(6-chloropyridazin-4-yl)-2-(furan-2-ylmethylene)-6-(3-hydroxyphenyl)imidazo[1,2-a]pyrazin-3(2H)-one

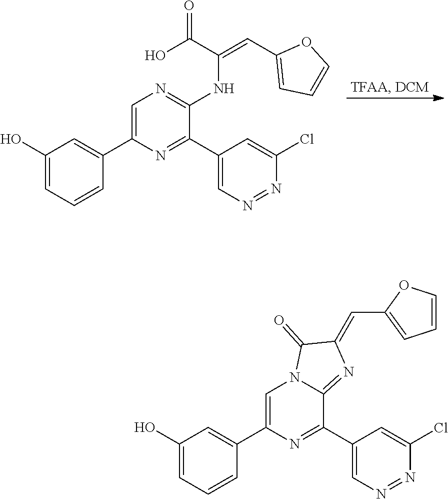

To a 20 mL vial, (Z)-2-((3-(6-chloropyridazin-4-yl)-5-(3-hydroxyphenyl)pyrazin-2-yl)amino)-3-(furan-2-yl)acrylic acid (3.5 mg, 0.0080 mmol), DCM (1 mL), and TFAA (0.5 mL) were added. The mixture was stirred for 10 min. The solvents were evaporated to afford (Z)-8-(6-chloropyridazin-4-yl)-2-(furan-2-ylmethylene)-6-(3-hydroxyphenyl)imidazo[1,2-a]pyrazin-3(2H)-one. LCMS (C₂₁H₁₂ClN₅O₃) (ES, m/z) 418 [M+H]⁺.

### Step 5: 8-(6-chloropyridazin-4-yl)-2-(furan-2-ylmethyl)-6-(3-hydroxyphenyl)imidazo[1,2-a]pyrazin-3(7H)-one

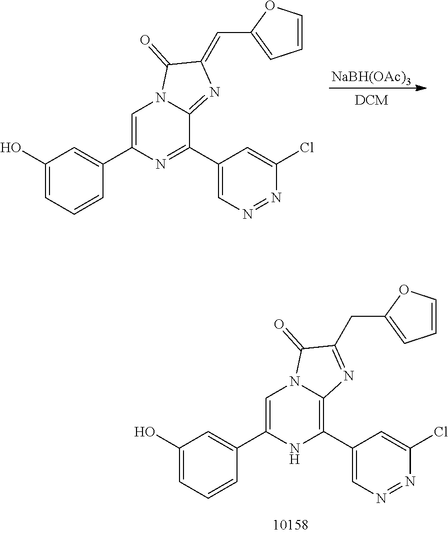 [SMILES: O=C1/C(=C/c2ccco2)N=C2C(c3cnnc(Cl)c3)=NC(c3cccc(O)c3)=CN12 ; O=c1c(Cc2ccco2)nc2c(-c3cnnc(Cl)c3)[nH]c(-c3cccc(O)c3)cn1-2 ; "NaBH(OAc)3"]

To a 20 mL vial, (Z)-8-(6-chloropyridazin-4-yl)-2-(furan-2-ylmethylene)-6-(3-hydroxyphenyl)imidazo[1,2-a]pyrazin-3(2H)-one (3.4 mg 0.0081 mmol) and DCM (2 mL) were added. The mixture was degassed with nitrogen for 1 min. To the mixture, sodium triacetoxy borohydride (25.9 mg, 0.122 mmol) was added. The mixture was stirred and heated at 50° C. for 30 min. The mixture was cooled to RT, diluted in 2% TFA in DMF, and filtered through a disc filter. The DCM was removed in vacuo. The mixture was then purified by reversed phase HPLC (MeCN/water w/0.1% TFA) to afford 10158. LCMS (C₂₁H₁₄ClN₅O₃) (ES, m/z) 420 [M+H]⁺. ¹H NMR (400 MHz, Methanol-d₄) δ 10.41 (s, 1H), 9.17 (s, 1H), 8.53 (s, 1H), 7.68-7.52 (m, 2H), 7.46-7.24 (m, 2H), 6.89 (dd, J=8.1, 2.5 Hz, 1H), 6.34 (dd, J=3.2, 1.9 Hz, 1H), 6.14 (d, J=3.2 Hz, 1H), 4.26 (s, 2H).

### Synthesis of Compound 10160: 6-(3-amino-2-fluorophenyl)-8-(6-chloropyridazin-4-yl)-2-(furan-2-ylmethyl)imidazo[1,2-a]pyrazin-3(7H)-one

### Step 1: Tert-butyl (Z)-2-((5-(3-amino-2-fluorophenyl)-3-bromopyrazin-2-yl)amino)-3-(furan-2-yl)acrylate

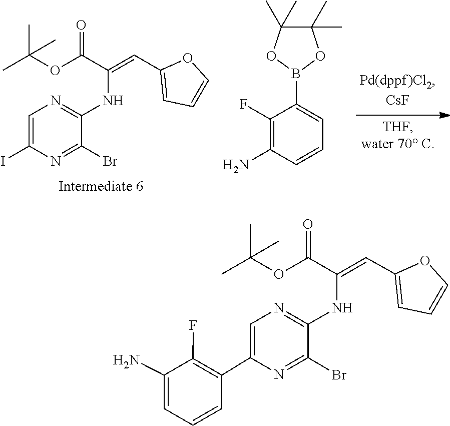 [SMILES: CC(C)(C)OC(=O)/C(=C/c1ccco1)Nc1ncc(I)nc1Br ; CC1(C)OB(c2cccc(N)c2F)OC1(C)C ; CC(C)(C)OC(=O)/C(=C/c1ccco1)Nc1ncc(-c2cccc(N)c2F)nc1Br]

To a 20 mL vial, Intermediate 6 (53.1 mg, 0.150 mmol), 2-fluoro-3-(4,4,5,5-tetramethyl-1,3,2-dioxaborolan-2-yl)aniline (51.2 mg, 0.216 mmol), CsF (82.0 mg, 0.539 mmol), Pd(dppf)Cl₂ (7.9 mg, 0.011 mmol), THF (1 mL), and water (0.2 mL) were added. The mixture was sparged with nitrogen for 2 min. The mixture was stirred and heated at 70° C. After 2 h, the mixture was adsorbed to Celite and purified by silica gel chromatography with 0-50% EtOAc in heptane as eluent to afford tert-butyl (Z)-2-((5-(3-amino-2-fluorophenyl)-3-bromopyrazin-2-yl)amino)-3-(furan-2-yl)acrylate. LCMS (C₂₁H₂₀BrFN₄O₃) (ES, m/z) 475 [M+H]⁺.

### Step 2: Tert-butyl (Z)-2-((5-(3-amino-2-fluorophenyl)-3-(6-chloropyridazin-4-yl)pyrazin-2-yl)amino)-3-(furan-2-yl)acrylate

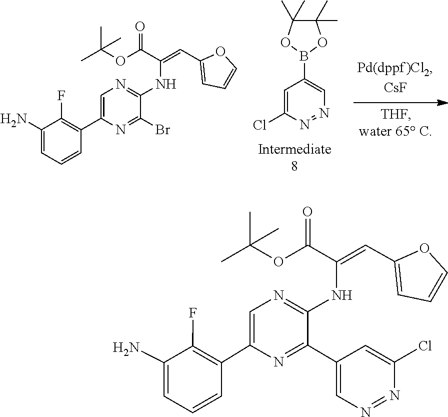 [SMILES: CC(C)(C)OC(=O)/C(=C/c1ccco1)Nc1ncc(-c2cccc(N)c2F)nc1Br ; CC1(C)OB(c2cnnc(Cl)c2)OC1(C)C ; CC(C)(C)OC(=O)/C(=C/c1ccco1)Nc1ncc(-c2cccc(N)c2F)nc1-c1cnnc(Cl)c1]

To a 20 mL vial, tert-butyl (Z)-2-((5-(3-amino-2-fluorophenyl)-3-bromopyrazin-2-yl)amino)-3-(furan-2-yl)acrylate (25.3 mg, 0.0532 mmol), Intermediate 8 (51.2 mg, 0.213 mmol), CsF (40.4 mg, 0.266 mmol), Pd(dppf)Cl₂ (3.9 mg, 0.0053 mmol), THF (1 mL), and water (0.3 mL) were added. The mixture was sparged with nitrogen for 2 min. The mixture was stirred and heated at 65° C. After 2 h, the mixture was adsorbed to Celite and purified by silica gel chromatography with 0-70% EtOAc in heptane as eluent to afford tert-butyl (Z)-2-((5-(3-amino-2-fluorophenyl)-3-(6-chloropyridazin-4-yl)pyrazin-2-yl)amino)-3-(furan-2-yl)acrylate. LCMS (C₂₅H₂₂ClFN₆O₃) (ES, m/z) 509 [M+H]⁺.

### Step 3: (Z)-2-((5-(3-amino-2-fluorophenyl)-3-(6-chloropyridazin-4-yl)pyrazin-2-yl)amino)-3-(furan-2-yl)acrylic acid

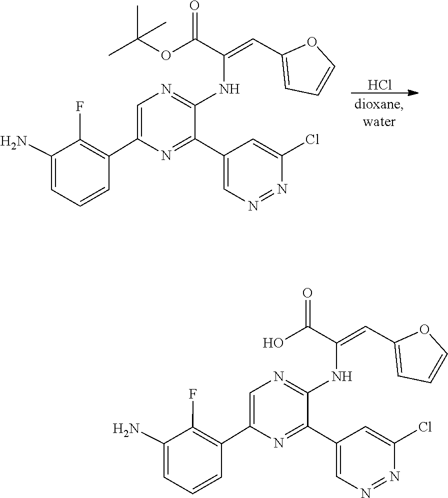 [SMILES: CC(C)(C)OC(=O)/C(=C/c1ccco1)Nc1ncc(-c2cccc(N)c2F)nc1-c1cnnc(Cl)c1 ; Cl ; Nc1cccc(-c2cnc(N/C(=C\c3ccco3)C(=O)O)c(-c3cnnc(Cl)c3)n2)c1F]

To a 20 mL vial, tert-butyl (Z)-2-((5-(3-amino-2-fluorophenyl)-3-(6-chloropyridazin-4-yl)pyrazin-2-yl)amino)-3-(furan-2-yl)acrylate (14.3 mg, 0.0281 mmol), 4 M HCl in dioxane (1 mL), and 6 M aqueous HCl (1 mL) were added. The mixture was stirred and heated at 50° C. for 30 min. The solvents were evaporated and the residue was co-evaporated with toluene to afford the (Z)-2-((5-(3-amino-2-fluorophenyl)-3-(6-chloropyridazin-4-yl)pyrazin-2-yl)amino)-3-(furan-2-yl)acrylic acid. LCMS (C₂₁H₁₄ClFN₆O₃) (ES, m/z) 453 [M+H]⁺.

### Step 4: (Z)-6-(3-amino-2-fluorophenyl)-8-(6-chloropyridazin-4-yl)-2-(furan-2-ylmethylene)imidazo[1,2-a]pyrazin-3(2H)-one

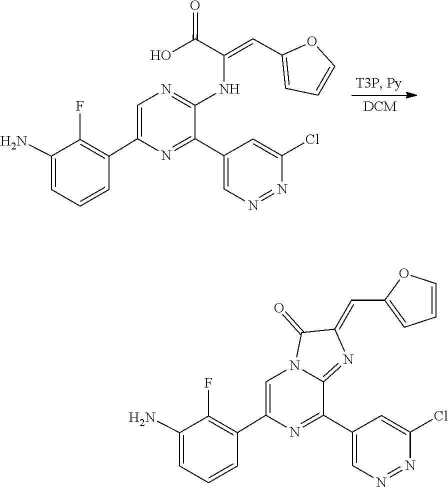 [SMILES: Nc1cccc(-c2cnc(N/C(=C\c3ccco3)C(=O)O)c(-c3cnnc(Cl)c3)n2)c1F ; "T3P, Py" ; Nc1cccc(C2=CN3C(=O)/C(=C/c4ccco4)N=C3C(c3cnnc(Cl)c3)=N2)c1F]

To a 20 mL vial, (Z)-2-((5-(3-amino-2-fluorophenyl)-3-(6-chloropyridazin-4-yl)pyrazin-2-yl)amino)-3-(furan-2-yl)acrylic acid (12.7 mg, 0.0285 mmol), DCM (3 mL), and pyridine (0.024 mL, 0.28 mmol) were added. The mixture was sonicated to form a suspension. To the stirring mixture, the T3P (50% in EtOAc, 0.086 mL, 0.140 mmol) was added. After 30 min, the mixture was quenched with saturated NaHCO₃ (4 mL). The organic layer was collected with a phase separator, and the remaining aqueous was washed 3 times with DCM. The combined organic layer was concentrated to afford (Z)-6-(3-amino-2-fluorophenyl)-8-(6-chloropyridazin-4-yl)-2-(furan-2-ylmethylene)imidazo[1,2-a]pyrazin-3(2H)-one. LCMS (C₂₁H₁₂ClFN₆O₂) (ES, m/z) 435 [M+H]⁺.

### Step 5: 6-(3-amino-2-fluorophenyl)-8-(6-chloropyridazin-4-yl)-2-(furan-2-ylmethyl)imidazo[1,2-a]pyrazin-3(7H)-one

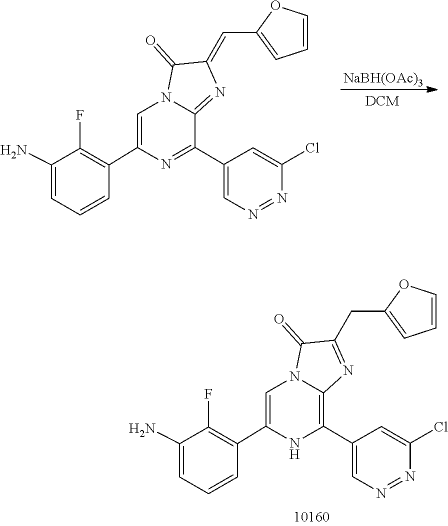 [SMILES: Nc1cccc(C2=CN3C(=O)/C(=C/c4ccco4)N=C3C(c3cnnc(Cl)c3)=N2)c1F ; Nc1cccc(-c2cn3c(=O)c(Cc4ccco4)nc-3c(-c3cnnc(Cl)c3)[nH]2)c1F ; "NaBH(OAc)3"]

To a 20 mL vial, (Z)-6-(3-amino-2-fluorophenyl)-8-(6-chloropyridazin-4-yl)-2-(furan-2-ylmethylene)imidazo[1,2-a]pyrazin-3(2H)-one (38.6 mg 0.089 mmol) and DCM (5 mL) were added. The mixture was degassed with nitrogen for 1 min. To the mixture, sodium triacetoxy borohydride (282 mg, 1.33 mmol) was added. The mixture was stirred and heated at 50° C. for 30 min. The mixture was cooled to RT and diluted in 0.2% TFA in 1:1 water:DMF. The DCM was removed in vacuo. The mixture was then purified by reversed phase HPLC (MeCN/water w/0.1% TFA) to afford 10160. LCMS (C₂₁H₁₄ClFN₆O₂) (ES, m/z) 420 [M+H]⁺. ¹H NMR (400 MHz, Methanol-d₄) δ 10.41 (s, 1H), 9.17 (s, 1H), 8.53 (s, 1H), 7.68-7.52 (m, 2H), 7.46-7.24 (m, 2H), 6.89 (dd, J=8.1, 2.5 Hz, 1H), 6.34 (dd, J=3.2, 1.9 Hz, 1H), 6.14 (d, J=3.2 Hz, 1H), 4.26 (s, 2H).

### Synthesis of Compound 10427:

### Step 1: tert-butyl 2-((3-bromo-5-(2,3-difluorophenyl)pyrazin-2-yl)amino)-3-(furan-2-yl)propanoate

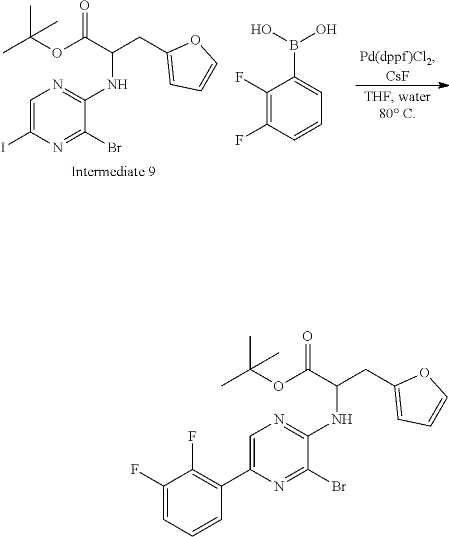

To a 20 mL vial, Intermediate 9 (100 mg, 0.202 mmol), (2,3-difluorophenyl)boronic acid (47.9 mg, 0.304 mmol), Pd(dppf)Cl₂ (7.4 mg, 0.010 mmol), THF (4 mL), and 1 M aqueous CsF (1.01 mL, 1.01 mmol) were added. The mixture was sparged with N₂ for 1 min. The mixture was stirred and heated at 80° C. After 2 h, the mixture was adsorbed to Celite and purified by silica gel chromatography with 0-10% EtOAc in heptane to afford tert-butyl 2-((3-bromo-5-(2,3-difluorophenyl)pyrazin-2-yl)amino)-3-(furan-2-yl)propanoate. LCMS (C₂₁H₂₀BrF₂N₃O₃) (ES, m/z) 480 [M+H]⁺.

### Step 2: Tert-butyl 2-((3-(6-chloropyridazin-4-yl)-5-(2,3-difluorophenyl)pyrazin-2-yl)amino)-3-(furan-2-yl)propanoate

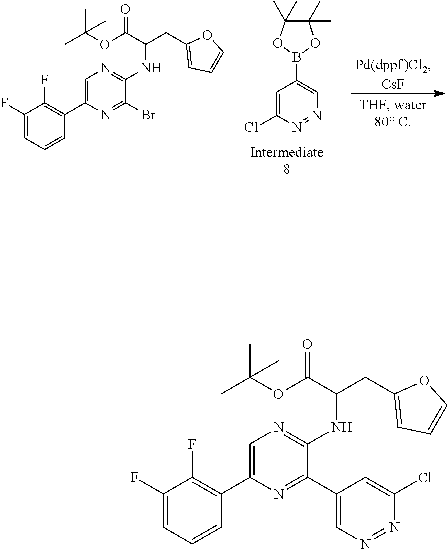 [SMILES: CC(C)(C)OC(=O)C(Cc1ccco1)Nc1ncc(-c2cccc(F)c2F)nc1Br ; CC1(C)OB(c2cnnc(Cl)c2)OC1(C)C ; "Pd(dppf)Cl2,CsF" ; CC(C)(C)OC(=O)C(Cc1ccco1)Nc1ncc(-c2cccc(F)c2F)nc1-c1cnnc(Cl)c1]

To a 20 mL vial, tert-butyl 2-((3-bromo-5-(2,3-difluorophenyl)pyrazin-2-yl)amino)-3-(furan-2-yl)propanoate (36.0 mg, 0.0750 mmol), Pd(dppf)Cl₂ (16.5 mg, 0.0225 mmol), Intermediate 8 (144 mg, 0.600 mmol), THF (4 mL), and 1 M aqueous CsF (0.245 mL, 0.245 mmol) were added. The mixture was sparged with N₂ for 1 min. The mixture was stirred and heated at 80° C. After 14 h, the mixture was adsorbed to Celite and purified by silica gel chromatography with 0-30% EtOAc in heptane to afford tert-butyl 2-((3-(6-chloropyridazin-4-yl)-5-(2,3-difluorophenyl)pyrazin-2-yl)amino)-3-(furan-2-yl)propanoate. LCMS (C₂₅H₂₂ClF₂N₅O₃) (ES, m/z) 514 [M+H]⁺.

### Step 3: 2-((3-(6-chloropyridazin-4-yl)-5-(2,3-difluorophenyl)pyrazin-2-yl)amino)-3-(furan-2-yl)propanoic acid

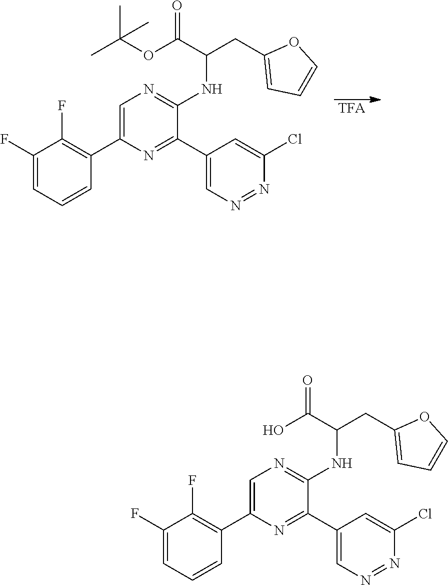 [SMILES: CC(C)(C)OC(=O)C(Cc1ccco1)Nc1ncc(-c2cccc(F)c2F)nc1-c1cnnc(Cl)c1 ; O=C(O)C(Cc1ccco1)Nc1ncc(-c2cccc(F)c2F)nc1-c1cnnc(Cl)c1 ; "TFA"]

To a 20 mL vial, tert-butyl 2-((3-(6-chloropyridazin-4-yl)-5-(2,3-difluorophenyl)pyrazin-2-yl)amino)-3-(furan-2-yl)propanoate (11.4 mg, 0.0222 mmol) and TFA (1 mL) were added. The mixture was stirred and heated at 35° C. for 1 h. The solvents were evaporated, and the residue was co-evaporated with toluene to afford 2-((3-(6-chloropyridazin-4-yl)-5-(2,3-difluorophenyl)pyrazin-2-yl)amino)-3-(furan-2-yl)propanoic acid. LCMS (C₂₁H₁₄ClF₂N₅O₃) (ES, m/z) 458 [M+H]⁺.

### Step 4: 8-(6-chloropyridazin-4-yl)-6-(2,3-difluorophenyl)-2-(furan-2-ylmethyl)imidazo[1,2-a]pyrazin-3(7H)-one

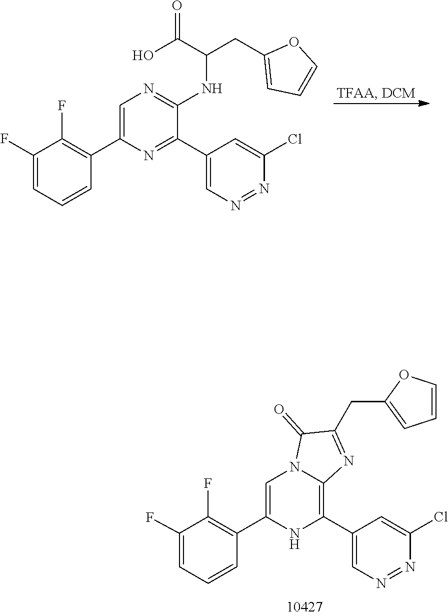

To a 20 mL vial, 2-((3-(6-chloropyridazin-4-yl)-5-(2,3-difluorophenyl)pyrazin-2-yl)amino)-3-(furan-2-yl)propanoic acid (10.2 mg, 0.0223 mmol) and DCM (1 mL) were added. The mixture was degassed with N₂ for 1 min. To the degassing mixture was added TFAA (0.5 mL). The mixture was stirred for 5 min. The solvents were evaporated, and the residue was purified by silica gel chromatography with 0-20% MeOH in DCM as eluent to afford 10427. LCMS (C₂₁H₁₂ClF₂N₅O₂) (ES, m/z) 440 [M+H]⁺. ¹H NMR (400 MHz, Methanol-d₄) δ 10.42 (s, 1H), 9.19 (s, 1H), 8.63 (s, 1H), 8.08 (d, J=8.4 Hz, 1H), 7.40 (d, J=1.9 Hz, 1H), 7.35 (dd, J=7.8, 5.0 Hz, 2H), 6.34 (dd, J=3.2, 1.9 Hz, 1H), 6.14 (d, J=3.2 Hz, 1H), 4.28 (s, 2H).

The compounds in the following table were prepared analogously to 10427 from Intermediates 9-13.

| Compound | Structure | LCMS (ES, m/z) [M + H]⁺ |
|---|---|---|
| 10299 | 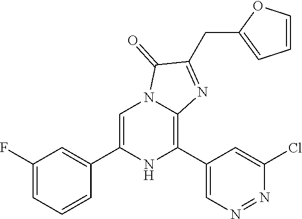 [SMILES: O=c1c(Cc2ccco2)nc2c(-c3cnnc(Cl)c3)[nH]c(-c3cccc(F)c3)cn1-2] | 422 |
| 10300 | 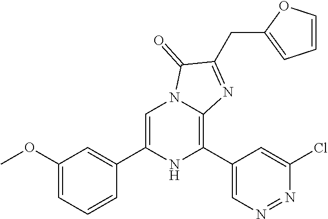 [SMILES: COc1cccc(-c2cn3c(=O)c(Cc4ccco4)nc-3c(-c3cnnc(Cl)c3)[nH]2)c1] | 434 |

### Synthesis of Model Compound 9812:

### Step 1: Tert-butyl (Z)-2-((3-(5-chloropyridin-3-yl)-5-phenylpyrazin-2-yl)amino)-3-(furan-2-yl)acrylate

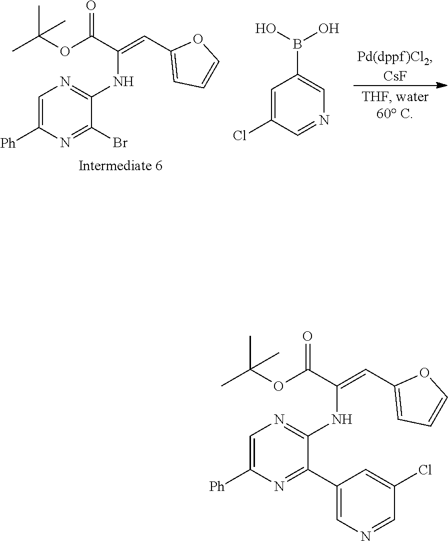 [SMILES: CC(C)(C)OC(=O)/C(=C/c1ccco1)Nc1ncc(-c2ccccc2)nc1Br ; OB(O)c1cncc(Cl)c1 ; "Pd(dppf)Cl2,CsF" ; CC(C)(C)OC(=O)/C(=C/c1ccco1)Nc1ncc(-c2ccccc2)nc1-c1cncc(Cl)c1]

To a 20 mL vial, Intermediate 6 (40.0 mg, 0.0904 mmol), (5-chloropyridin-3-yl)boronic acid (21.4 mg, 0.136 mmol), CsF (27.5 mg, 0.181 mmol), Pd(dppf)Cl₂ (3.3 mg, 0.0045 mmol), THF (2 mL), and water (0.3 mL) were added. The mixture was sparged with nitrogen for 2 min. The mixture was stirred and heated at 60° C. After 14 h, the mixture was adsorbed to Celite and purified by silica gel chromatography with 0-60% EtOAc in heptane as eluent to tert-butyl (Z)-2-((3-(5-chloropyridin-3-yl)-5-phenylpyrazin-2-yl)amino)-3-(furan-2-yl)acrylate. LCMS (C₂₆H₂₃ClN₄O₃) (ES, m/z) 475 [M+H]⁺.

### Step 2: (Z)-2-((3-(5-chloropyridin-3-yl)-5-phenylpyrazin-2-yl)amino)-3-(furan-2-yl)acrylic acid

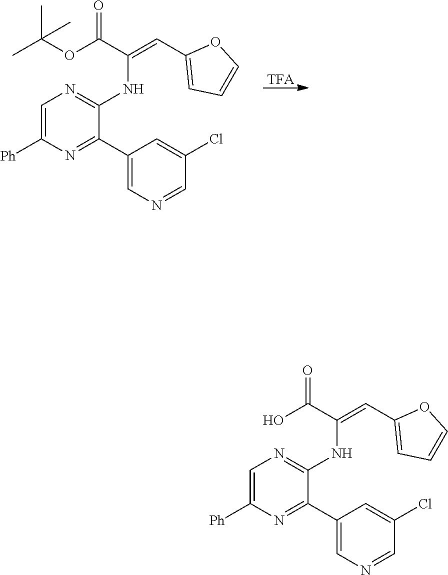 [SMILES: CC(C)(C)OC(=O)/C(=C/c1ccco1)Nc1ncc(-c2ccccc2)nc1-c1cncc(Cl)c1 ; O=C(O)/C(=C/c1ccco1)Nc1ncc(-c2ccccc2)nc1-c1cncc(Cl)c1 ; "TFA"]

To a 20 mL vial, tert-butyl (Z)-2-((3-(5-chloropyridin-3-yl)-5-phenylpyrazin-2-yl)amino)-3-(furan-2-yl)acrylate (31.9 mg, 0.0672 mmol) and TFA (1 mL) were added. The mixture was stirred for 1 h. The solvents were evaporated to afford (Z)-2-((3-(5-chloropyridin-3-yl)-5-phenylpyrazin-2-yl)amino)-3-(furan-2-yl)acrylic acid. LCMS (C₂₂H₁₅ClN₄O₃) (ES, m/z) 419 [M+H]⁺.

### Step 3: (Z)-8-(5-chloropyridin-3-yl)-2-(furan-2-ylmethylene)-6-phenylimidazo[1,2-a]pyrazin-3(2H)-one

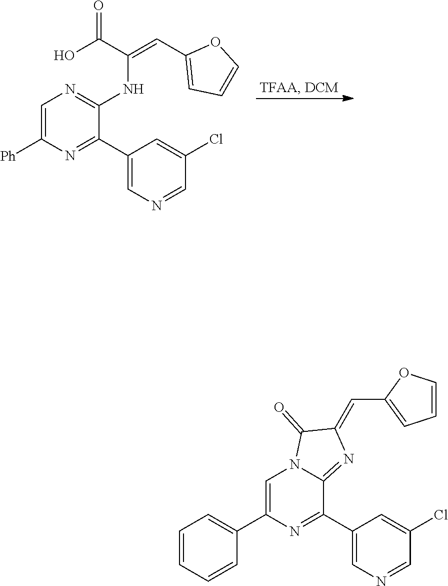

To a 20 mL vial, (Z)-2-((3-(5-chloropyridin-3-yl)-5-phenylpyrazin-2-yl)amino)-3-(furan-2-yl)acrylic acid (28.1 mg, 0.0671 mmol) DCM (1 mL) and TFAA (0.5 mL) were added. The mixture was stirred for 10 min. The solvents were evaporated to afford (Z)-8-(5-chloropyridin-3-yl)-2-(furan-2-ylmethylene)-6-phenylimidazo[1,2-a]pyrazin-3(2H)-one. LCMS (C₂₂H₁₃ClN₄O₂) (ES, m/z) 401 [M+H]⁺.

### Step 4: 8-(5-chloropyridin-3-yl)-2-(furan-2-ylmethyl)-6-phenylimidazo[1,2-a]pyrazin-3(7H)-one

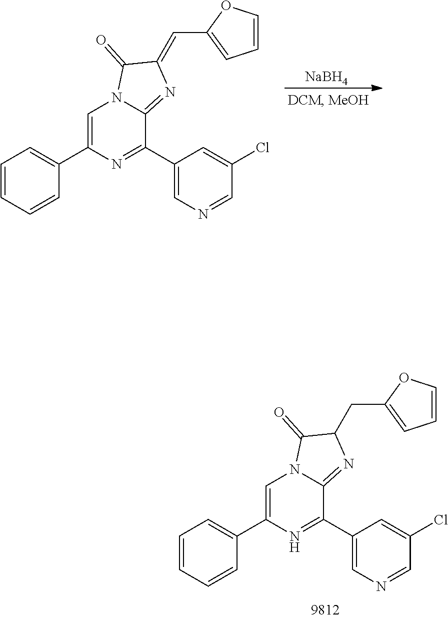 [SMILES: O=C1/C(=C/c2ccco2)N=C2C(c3cncc(Cl)c3)=NC(c3ccccc3)=CN12 ; "NaBH4" ; O=C1C(Cc2ccco2)N=C2C(c3cncc(Cl)c3)=NC(c3ccccc3)=CN12]

To a 20 mL vial, (Z)-8-(5-chloropyridin-3-yl)-2-(furan-2-ylmethylene)-6-phenylimidazo[1,2-a]pyrazin-3(2H)-one (26.9 mg 0.0671 mmol), DCM (0.5 mL), and MeOH (0.5 mL) were added. To the mixture, sodium borohydride (12.7 mg, 0.336 mmol) was added. The mixture was stirred for 10 min. The mixture was quenched with AcOH (0.1 mL), filtered, and the solvents of the filtrate were evaporated. The residue was purified by reversed phase HPLC (MeCN/water w/0.1% TFA) to afford 9812. LCMS (C₂₂H₁₅ClN₄O₂) (ES, m/z) 403 [M+H]⁺. ¹H NMR (400 MHz, Methanol-d₄) δ 9.57 (d, J=1.8 Hz, 1H), 8.97 (t, J=2.1 Hz, 1H), 8.77 (d, J=2.3 Hz, 1H), 8.46 (s, 1H), 8.25-8.04 (m, 2H), 7.64-7.51 (m, 2H), 7.51-7.45 (m, 1H), 7.42 (dd, J=1.9, 0.8 Hz, 1H), 6.34 (dd, J=3.2, 1.9 Hz, 1H), 6.15 (dd, J=3.1, 1.1 Hz, 1H), 4.25 (s, 2H).

### Example 3. Luminescent Properties

Experiments were conducted during the development of embodiments herein to test the effect of derivatization on the biochemical properties of novel coelenterazine analogues. Properties tested include the analogues' ability to be utilized by NANOLUC® luciferase, as assessed through the determination of Vₘₐₓ and K_{M}, as well as the peak wavelength of luminescence emission. The substitution of the phenyl ring present on compound 7314 for either of the chloropyridin groups found on compounds 9812 or 6356 were found to red-shift the peak emission when reacted with NANOLUC® luciferase. These data suggested that an analogue containing a heterocycle of two nitrogen may demonstrate more red emission. Compound 9894 was synthesized with a chloropyridazin ring and analyses demonstrated a far-red shift to 622 nm (Scheme 1).

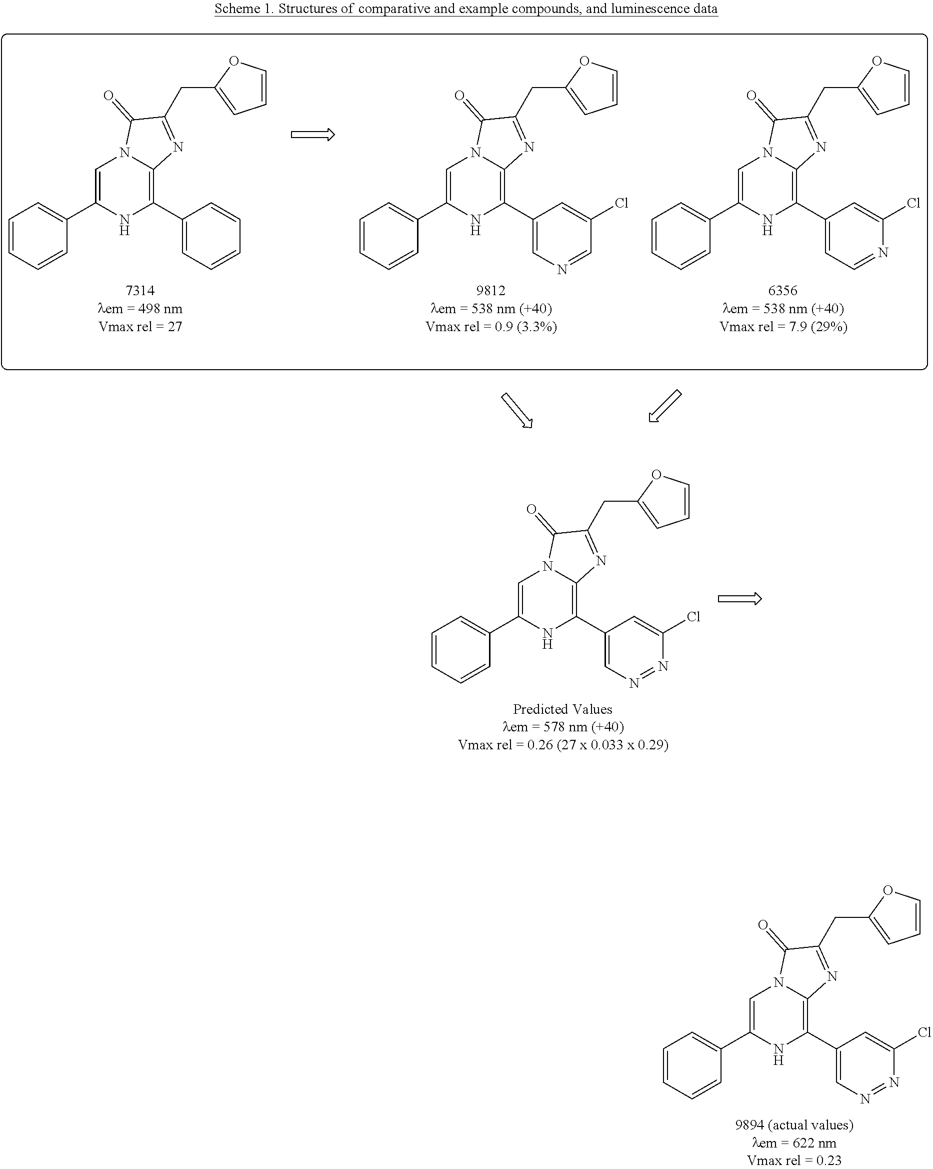

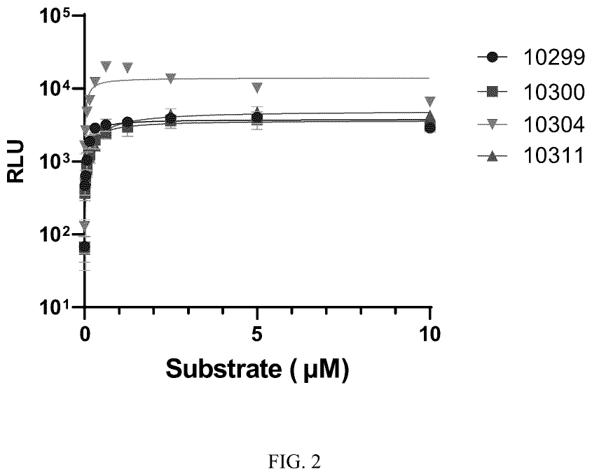

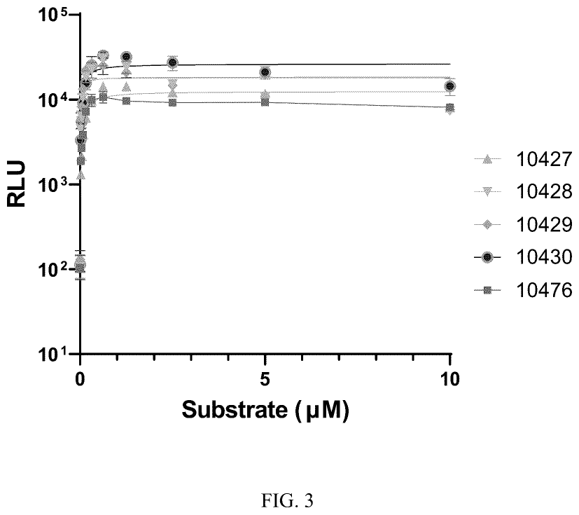

Data for additional compounds are presented in Tables 1 and 2, and in FIGS. 2 and 3.

TABLE 1
Data for Compounds 10299, 10300, 10304, and 10311

| | Vmax | Km | Vmax/Km | λ max | |
|---|---|---|---|---|---|
| Compound | (norm) | (μM) | (norm) | (nm) | |
| Furimazine | 1 | 0.23 | 1 | 466 | nm |
| 10299 | 0.000505 | 0.15 | 0.000808 | 622 | nm |
| 10300 | 0.000505 | 0.27 | 0.000404 | nd | |
| 10304 | 0.001818 | 0.13 | 0.003232 | nd | |
| 10311 | 0.000707 | 0.55 | 0.000303 | ~608 | nm |

TABLE 2
Data for Compounds 10427, 10428, 10429, 10430, and 10476

| | Vmax | Km | Vmax/Km | λ max |
|---|---|---|---|---|
| Compound | (norm) | (μM) | (norm) | (nm) |
| Furimazine | 1 | 0.56 | 1 | 462 nm |
| 10427 | 0.0015 | 0.16 | 0.005 | 600 nm |
| 10428 | 0.0023 | 0.05 | 0.026 | 612 nm |
| 10429 | 0.0022 | 0.03 | 0.041 | 598 nm |
| 10430 | 0.0033 | 0.11 | 0.017 | nd |
| 10476 | 0.0012 | 0.08 | 0.008 | 584 nm |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A compound of formula (I): 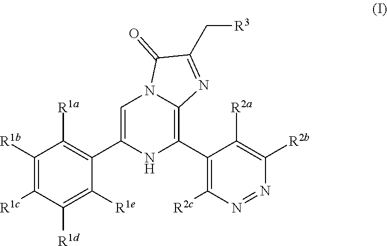
or a tautomer or a salt thereof, wherein:
R^{1a}, R^{1b}, R^{1c}, R^{1d}, R^{1e}, R^{2a}, R^{2b}, and R^{2c} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2}, NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀haloalkyl, hydroxy, cyano, nitro, amino, C₁₋₁₀alkoxy, aryl, heteroaryl, heterocycle, and C₃₋₆cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

2. The compound of claim 1, wherein the compound is a compound of formula (I′): 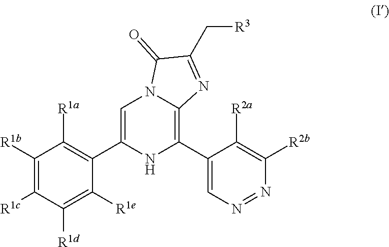
or a tautomer or a salt thereof, wherein:
R^{1a}, R^{1b}, R^{1c}, R^{1d}, R^{1e}, R^{2a}, and R^{2b} are each independently selected from hydrogen, halogen, —CN, —NO₂, C₁₋₁₀alkyl, C₁₋₁₀haloalkyl, C₁₋₄haloalkylene-OC₁₋₄alkyl, C₂₋₁₀alkenyl, C₂₋₁₀alkynyl, —C(O)OR^{x1}, —CONR^{x1}R^{x2}, —OC(O)NR^{x1}R^{x2}, —NR^{x3}C(O)OR^{x1}, —OR^{x1}, —C₁₋₁₀alkylene-OR^{x1}, —OC(O)R^{x1}, —NR^{x1}R^{x2}, —C₁₋₁₀alkylene-NR^{x1}R^{x2}, —NR^{x3}C(O)R^{x1}, —NR^{x3}C(O)NR^{x1}R^{x2}, —SO₂R^{x1}, —SO₂NR^{x1}R^{x2}, NR^{x3}SO₂R^{x1}, —SO₂OR^{x1}, —OSO₂R^{x1}, —OSO₃R^{x1}, —OP(O)(OH)OR^{x1}, and —OSi(C₁₋₁₀alkyl)₃; wherein R^{1a} and R^{1b}, together with the carbon atoms to which they are attached, are optionally taken together to form an optionally substituted ring; and wherein R^{2a} and R^{2b}, together with the carbon atoms to which they are attached, are optionally taken together to form an optionally substituted ring;
R³ is aryl or heteroaryl, wherein the aryl or heteroaryl is optionally substituted with 1, 2, or 3 substituents independently selected from halogen, C₁₋₁₀alkyl, C₂₋₁₀alkenyl, C₂₋₁₀alkynyl, C₁₋₁₀haloalkyl, hydroxy, cyano, nitro, amino, C₁₋₁₀alkoxy, aryl, heteroaryl, heterocycle, and C₃₋₆cycloalkyl;
R^{x1}, R^{x2}, and R^{x3}, at each occurrence, are independently selected from hydrogen, C₁₋₁₀alkyl, C₁₋₁₀haloalkyl, C₃₋₁₂cycloalkyl, and —C₁₋₃alkylene-C₃₋₁₂cycloalkyl, wherein each cycloalkyl is optionally substituted with 1, 2, 3, or 4 substituents independently selected from the group consisting of halogen, C₁₋₄alkyl, and C₁₋₄haloalkyl;
alternatively, R^{x1} and R^{x2}, together with the nitrogen atom to which they are attached, form a 4- to 8-membered saturated or partially unsaturated heterocyclic ring, optionally substituted with 1, 2, 3, or 4 substituents independently selected from the group consisting of C₁₋₄alkyl, C₁₋₄haloalkyl, oxo, —OH, and —OC₁₋₄alkyl.

3. The compound of claim 1, or a tautomer or a salt thereof, wherein R^{1a}, R^{1b}, R^{1c}, R^{1d}, R^{1e}, R^{2a}, R^{2b}, and R^{2c} are each independently selected from hydrogen, halogen, —CN, C₁₋₄alkyl, C₁₋₄haloalkyl, —OR^{x1}, and —NR^{x1}R^{x2}, wherein R^{x1} and R^{x2} are independently selected from hydrogen and C₁₋₄alkyl.

4. (canceled)

5. The compound of claim 1, or a tautomer or a salt thereof, wherein R^{1a} is hydrogen or halogen.

6. (canceled)

7. (canceled)

8. The compound of claim 7, wherein R^{1a} is fluoro.

9. The compound of claim 1, or a tautomer or a salt thereof, wherein R^{1b} is hydrogen, halogen, C₁₋₄alkyl, C₁₋₄haloalkyl, —OR^{x1}, or —NR^{x1}R^{x2}, wherein R^{x1} and R^{x2} are independently selected from hydrogen and methyl.

10. The compound of claim 1, or a tautomer or a salt thereof, wherein R^{1b} is hydrogen, methyl, fluoro, difluoromethyl, methoxy, —OH, or —NH₂.

11.-21. (canceled)

22. The compound of claim 1, or a tautomer or a salt thereof, wherein R^{2b} is hydrogen, halogen, —CN, or C₁₋₁₀alkyl.

23. (canceled)

24. The compound of claim 1, or a tautomer or a salt thereof, wherein R^{2b} is halogen.

25. (canceled)

26. The compound of claim 1, or a tautomer or a salt thereof, wherein R^{1c}, R^{1d}, R^{1e}, R^{2a}, and R^{2c} are each hydrogen.

27. (canceled)

28. The compound of claim 1, or a tautomer or a salt thereof, wherein R³ is phenyl or a monocyclic 5- or 6-membered heteroaryl having 1 or 2 heteroatoms independently selected from O, S, and N.

29. (canceled)

30. The compound of claim 1, or a tautomer or a salt thereof, wherein R³ is phenyl, furanyl, or pyridinyl.

31. (canceled)

32. (canceled)

33. The compound of claim 1, or a tautomer or a salt thereof, wherein R³ is unsubstituted or substituted with 1 or 2 substituents selected from C₁₋₄alkyl, C₁₋₄haloalkyl, C₁₋₄alkoxy, halo, and hydroxy.

34. The compound of claim 1, or a tautomer or a salt thereof, wherein R³ is unsubstituted or substituted with 1 substituent selected from fluoro and methoxy.

35.-37. (canceled)

38. The compound of claim 1, wherein the compound is a compound of formula (Ia): 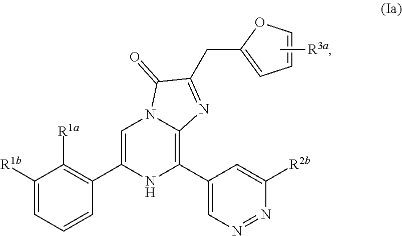
or a tautomer or a salt thereof, wherein:
R^{1a}, R^{1b}, and R^{2b} are each independently selected from hydrogen, halogen, —OR^{x1}, and —NR^{x1}R^{x2}, wherein R^{x1} and R^{x2} are each independently selected from hydrogen and C₁₋₄alkyl; and
R^{3a} is hydrogen, halogen, C₁₋₄alkyl, or C₁₋₄haloalkyl.

39.-44. (canceled)

45. The compound of claim 1, wherein the compound is a compound of formula (Ib): 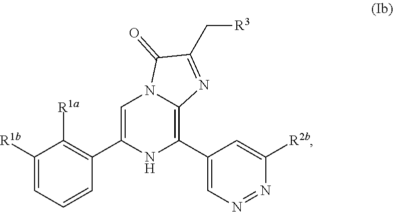
or a tautomer or a salt thereof, wherein:
R^{1a}, R^{1b}, and R^{2b} are each independently selected from hydrogen, C₁₋₄alkyl, C₁₋₄haloalkyl, halogen, —OR^{x1}, and —NR^{x1}R^{x2}, wherein R^{x1} and R^{x2} are each independently selected from hydrogen and C₁₋₄alkyl; and
R³ is phenyl or a monocyclic 5- or 6-membered heteroaryl having 1 or 2 heteroatoms independently selected from O, S, and N, wherein R³ is unsubstituted or substituted with 1 substituent selected from halo and C₁₋₄alkoxy.

46.-56. (canceled)

57. The compound of claim 1, wherein the compound is selected from the group consisting of: 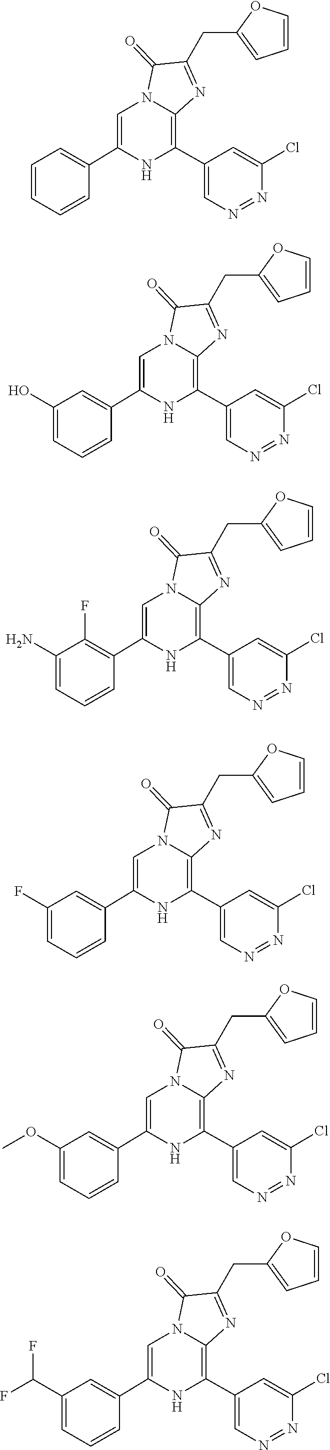 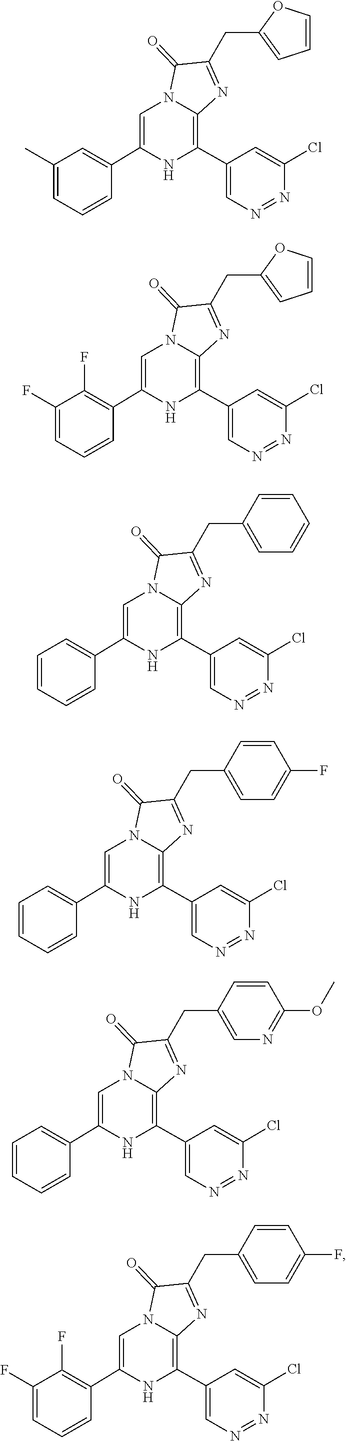
and tautomers and salts thereof.

58. A kit comprising the compound of claim 1, or a tautomer or a salt thereof, wherein the kit further comprises a luciferase, a buffer reagent, or instructions for performing a luminescence assay, or any combination thereof.

59.-61. (canceled)

62. A method for detecting luminescence in a sample, the method comprising:
contacting a sample with the compound of claim 1, or a tautomer or a salt thereof;
contacting the sample with a coelenterazine-utilizing luciferase, if it is not present in the sample; and
detecting luminescence.

63. (canceled)

64. (canceled)

65. A method for detecting luminescence in a transgenic animal comprising:
administering the compound of claim 1, or a tautomer or a salt thereof, to a transgenic animal; and
detecting luminescence;
wherein the transgenic animal expresses a coelenterazine-utilizing luciferase.
